SEQUENCE LISTING (126 sequences)

SEQ ID NO: 1 - DNA, 3 bp, Artificial Sequence
  Synthetic
  misc_feature (1)..(1): n is a, c, g, or t
  misc_feature (2)..(2): r is a, or g
nrg

SEQ ID NO: 2 - DNA, 3 bp, Artificial Sequence
  Synthetic
  misc_feature (1)..(1): n is a, c, g, or t
ngg

SEQ ID NO: 3 - DNA, 3 bp, Artificial Sequence
  Synthetic
  misc_feature (1)..(1): n is a, c, g, or t
nag

SEQ ID NO: 4 - DNA, 5 bp, Artificial Sequence
  Synthetic
  misc_feature (1)..(1): n is a, c, g, or t
  misc_feature (4)..(4): n is a, c, g, or t
nggng

SEQ ID NO: 5 - DNA, 7 bp, Artificial Sequence
  Synthetic
  misc_feature (1)..(2): n is a, c, g, or t
  misc_feature (7)..(7): w is a, or t
nnagaaw

SEQ ID NO: 6 - DNA, 4 bp, Artificial Sequence
  Synthetic
  misc_feature (1)..(1): n is a, c, g, or t
  misc_feature (4)..(4): r is a, or g
naar

SEQ ID NO: 7 - DNA, 5 bp, Artificial Sequence
  Synthetic
  misc_feature (1)..(2): n is a, c, g, or t
  misc_feature (4)..(5): r is a, or g
nngrr

SEQ ID NO: 8 - DNA, 6 bp, Artificial Sequence
  Synthetic
  misc_feature (1)..(2): n is a, c, g, or t
  misc_feature (4)..(5): r is a, or g
  misc_feature (6)..(6): n is a, c, g, or t
nngrrn

SEQ ID NO: 9 - DNA, 6 bp, Artificial Sequence
  Synthetic
  misc_feature (1)..(2): n is a, c, g, or t
  misc_feature (4)..(5): r is a, or g
nngrrt

SEQ ID NO: 10 - DNA, 6 bp, Artificial Sequence
  Synthetic
  misc_feature (1)..(2): n is a, c, g, or t
  misc_feature (4)..(5): r is a, or g
  misc_feature (6)..(6): v is a, c, or g
nngrrv

SEQ ID NO: 11 - DNA, 8 bp, Artificial Sequence
  Synthetic
  misc_feature (1)..(4): n is a, c, g, or t
nnnngatt

SEQ ID NO: 12 - DNA, 8 bp, Artificial Sequence
  Synthetic
  misc_feature (1)..(4): n is a, c, g, or t
  misc_feature (6)..(8): n is a, c, g, or t
nnnngnnn

SEQ ID NO: 13 - DNA, 3 bp, Artificial Sequence
  Synthetic
  misc_feature (1)..(1): n is a, c, g, or t
nga

SEQ ID NO: 14 - DNA, 6 bp, Artificial Sequence
  Synthetic
  misc_feature (1)..(3): n is a, c, g, or t
  misc_feature (4)..(5): r is a, or g
nnnrrt

SEQ ID NO: 15 - DNA, 6 bp, Artificial Sequence
  Synthetic
attcct

SEQ ID NO: 16 - DNA, 4 bp, Artificial Sequence
  Synthetic
  misc_feature (1)..(1): n is a, c, g, or t
  misc_feature (4)..(4): n is a, c, g, or t
ngan

SEQ ID NO: 17 - DNA, 4 bp, Artificial Sequence
  Synthetic
  misc_feature (1)..(1): n is a, c, g, or t
  misc_feature (3)..(3): n is a, c, g, or t
ngng

SEQ ID NO: 18 - DNA, 86 bp, Artificial Sequence
  Synthetic
gtttaagagctatgctggaaacagcatagcaagtttaaataaggctagtccgttatcaacttgaaaaagtggcaccgagtcggtgc

SEQ ID NO: 19 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 20 - protein, 1368 aa, Streptococcus pyogenes [sequence omitted]

SEQ ID NO: 21 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 22 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 24 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 25 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 26 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 27 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 28 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 29 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 30 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 31 - DNA, 3242 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 32 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 33 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 34 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 35 - protein, 2414 aa, Artificial Sequence
  Synthetic
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 36 - protein, 617 aa, Artificial Sequence
  Synthetic
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 37 - protein, 1497 aa, Artificial Sequence
  Synthetic
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 38 - DNA, 4491 bp, Artificial Sequence
  Synthetic
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 39 - DNA, 4560 bp, Artificial Sequence
  Synthetic
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 40 - protein, 1519 aa, Artificial Sequence
  Synthetic
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 41 - DNA, 3570 bp, Artificial Sequence
  Synthetic
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 42 - protein, 1190 aa, Artificial Sequence
  Synthetic
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 43 - DNA, 2154 bp, Artificial Sequence
  Synthetic
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 44 - protein, 718 aa, Artificial Sequence
  Synthetic
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 45 - DNA, 21 bp, Artificial Sequence
  Synthetic
agaggcacgtggttaagctct

SEQ ID NO: 46 - DNA, 21 bp, Artificial Sequence
  Synthetic
aaaggaactgaagtgagactg

SEQ ID NO: 47 - DNA, 21 bp, Artificial Sequence
  Synthetic
gactggtggagtccacacccc

SEQ ID NO: 48 - DNA, 21 bp, Artificial Sequence
  Synthetic
tggtttcctgtatagcccccc

SEQ ID NO: 49 - DNA, 21 bp, Artificial Sequence
  Synthetic
cttcatgcaaaaggaactgaa

SEQ ID NO: 50 - DNA, 21 bp, Artificial Sequence
  Synthetic
gagactggtggagtccacacc

SEQ ID NO: 51 - DNA, 21 bp, Artificial Sequence
  Synthetic
gttccttttgcatgaagagct

SEQ ID NO: 52 - DNA, 21 bp, Artificial Sequence
  Synthetic
aaggaactgaagtgagactgg

SEQ ID NO: 53 - DNA, 21 bp, Artificial Sequence
  Synthetic
cacggagcgagacatctcggc

SEQ ID NO: 54 - DNA, 21 bp, Artificial Sequence
  Synthetic
acctttggcctacggcgacgg

SEQ ID NO: 55 - DNA, 21 bp, Artificial Sequence
  Synthetic
gagcacagctaaggccacgga

SEQ ID NO: 56 - DNA, 21 bp, Artificial Sequence
  Synthetic
agaaggcatgcactagactgg

SEQ ID NO: 57 - DNA, 21 bp, Artificial Sequence
  Synthetic
ggagaggaaggaccagagcgg

SEQ ID NO: 58 - RNA, 21 nt, Artificial Sequence
  Synthetic
agaggcacgugguuaagcucu

SEQ ID NO: 59 - RNA, 21 nt, Artificial Sequence
  Synthetic
aaaggaacugaagugagacug

SEQ ID NO: 60 - RNA, 21 nt, Artificial Sequence
  Synthetic
gacugguggaguccacacccc

SEQ ID NO: 61 - RNA, 21 nt, Artificial Sequence
  Synthetic
ugguuuccuguauagcccccc

SEQ ID NO: 62 - RNA, 21 nt, Artificial Sequence
  Synthetic
cuucaugcaaaaggaacugaa

SEQ ID NO: 63 - RNA, 21 nt, Artificial Sequence
  Synthetic
gagacugguggaguccacacc

SEQ ID NO: 64 - RNA, 21 nt, Artificial Sequence
  Synthetic
guuccuuuugcaugaagagcu

SEQ ID NO: 65 - RNA, 21 nt, Artificial Sequence
  Synthetic
aaggaacugaagugagacugg

SEQ ID NO: 66 - RNA, 21 nt, Artificial Sequence
  Synthetic
cacggagcgagacaucucggc

SEQ ID NO: 67 - RNA, 21 nt, Artificial Sequence
  Synthetic
accuuuggccuacggcgacgg

SEQ ID NO: 68 - RNA, 21 nt, Artificial Sequence
  Synthetic
gagcacagcuaaggccacgga

SEQ ID NO: 69 - RNA, 21 nt, Artificial Sequence
  Synthetic
agaaggcaugcacuagacugg

SEQ ID NO: 70 - RNA, 21 nt, Artificial Sequence
  Synthetic
ggagaggaaggaccagagcgg

SEQ ID NO: 71 - DNA, 8757 bp, Artificial Sequence
  Synthetic
gtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaatgaacaaaagctggagctccaccgcgggcggccgcaaaaaaatctcgccaacaagttgacgagataaacacggcattttgccttgttttagtagattctgtttccagagtactaaaacagagacgtccgtctccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggcattaacccgtgtcggctccagatctggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagagccaccatgtacccatacgatgttccagattacgctatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagttgctagcgagggcagaggaagtcttctaacatgcggtgacgtggaggagaatcccggccctggtaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaccggttgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtact

SEQ ID NO: 72 - DNA, 8526 bp, Artificial Sequence
  Synthetic
gtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaatgaacaaaagctggagctccaccgcgggcggccgcaaaaaaatctcgccaacaagttgacgagataaacacggcattttgccttgttttagtagattctgtttccagagtactaaaacagagacgtccgtctccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggcattaacccgtgtcggctccagatctggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagagccaccatgtacccatacgatgttccagattacgctatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagttgctagcgagggcagaggaagtcttctaacatgcggtgacgtggaggagaatcccggccctatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtttcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtgaaccggttgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtact

SEQ ID NO: 73 - protein, 7 aa, Artificial Sequence
  Synthetic
PKKKRKV

SEQ ID NO: 74 - protein, 5 aa, Artificial Sequence
  Synthetic
  REPEAT (1)..(5): repeat between 0 and 10
GGGGS

SEQ ID NO: 75 - protein, 5 aa, Artificial Sequence
  Synthetic
GGGGG

SEQ ID NO: 76 - protein, 5 aa, Artificial Sequence
  Synthetic
GGAGG

SEQ ID NO: 77 - protein, 7 aa, Artificial Sequence
  Synthetic
GGGGSSS

SEQ ID NO: 78 - protein, 7 aa, Artificial Sequence
  Synthetic
GGGGAAA

SEQ ID NO: 79 - protein, 378 aa, Artificial Sequence
  Synthetic
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 80 - DNA, 1134 bp, Artificial Sequence
  Synthetic
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 81 - protein, 522 aa, Artificial Sequence
  Synthetic
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 82 - DNA, 1566 bp, Artificial Sequence
  Synthetic
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 83 - DNA, 21 bp, Artificial Sequence
  Synthetic
agtggataaaaggctgtgtgg

SEQ ID NO: 84 - DNA, 21 bp, Artificial Sequence
  Synthetic
cagtgaaagagaaaaggaaca

SEQ ID NO: 85 - DNA, 21 bp, Artificial Sequence
  Synthetic
gtttcttccaaaggtaagcat

SEQ ID NO: 86 - DNA, 21 bp, Artificial Sequence
  Synthetic
gagagtcactttcagggaaag

SEQ ID NO: 87 - DNA, 21 bp, Artificial Sequence
  Synthetic
agagaggctaagtagatcact

SEQ ID NO: 88 - DNA, 21 bp, Artificial Sequence
  Synthetic
gactgcacctataaaaagcaa

SEQ ID NO: 89 - DNA, 21 bp, Artificial Sequence
  Synthetic
tttggcaaaggagcacatcag

SEQ ID NO: 90 - DNA, 21 bp, Artificial Sequence
  Synthetic
taaatgttcacaagccaatag

SEQ ID NO: 91 - DNA, 21 bp, Artificial Sequence
  Synthetic
ggacaatctctgagaatgagg

SEQ ID NO: 92 - DNA, 21 bp, Artificial Sequence
  Synthetic
atagagactggatggacccac

SEQ ID NO: 93 - DNA, 21 bp, Artificial Sequence
  Synthetic
gtgggtccatccagtctctat

SEQ ID NO: 94 - DNA, 21 bp, Artificial Sequence
  Synthetic
tagatggttccaagaagggag

SEQ ID NO: 95 - DNA, 21 bp, Artificial Sequence
  Synthetic
tctcacccagcacttcataag

SEQ ID NO: 96 - DNA, 21 bp, Artificial Sequence
  Synthetic
agattcccctgccgttgaagg

SEQ ID NO: 97 - DNA, 21 bp, Artificial Sequence
  Synthetic
tcaattgctggaggtgtgggc

SEQ ID NO: 98 - DNA, 21 bp, Artificial Sequence
  Synthetic
actcagcttatgaagtgctgg

SEQ ID NO: 99 - DNA, 21 bp, Artificial Sequence
  Synthetic
gtgctgggtgagaccactgcc

SEQ ID NO: 100 - DNA, 21 bp, Artificial Sequence
  Synthetic
ttttatgggcgtagctgaaga

SEQ ID NO: 101 - DNA, 21 bp, Artificial Sequence
  Synthetic
tcgggaggagcagaggaggag

SEQ ID NO: 102 - RNA, 21 nt, Artificial Sequence
  Synthetic
aguggauaaaaggcugugugg

SEQ ID NO: 103 - RNA, 21 nt, Artificial Sequence
  Synthetic
cagugaaagagaaaaggaaca

SEQ ID NO: 104 - RNA, 21 nt, Artificial Sequence
  Synthetic
guuucuuccaaagguaagcau

SEQ ID NO: 105 - RNA, 21 nt, Artificial Sequence
  Synthetic
gagagucacuuucagggaaag

SEQ ID NO: 106 - RNA, 21 nt, Artificial Sequence
  Synthetic
agagaggcuaaguagaucacu

SEQ ID NO: 107 - RNA, 21 nt, Artificial Sequence
  Synthetic
gacugcaccuauaaaaagcaa

SEQ ID NO: 108 - RNA, 21 nt, Artificial Sequence
  Synthetic
uuuggcaaaggagcacaucag

SEQ ID NO: 109 - RNA, 21 nt, Artificial Sequence
  Synthetic
uaaauguucacaagccaauag

SEQ ID NO: 110 - RNA, 21 nt, Artificial Sequence
  Synthetic
ggacaaucucugagaaugagg

SEQ ID NO: 111 - RNA, 21 nt, Artificial Sequence
  Synthetic
auagagacuggauggacccac

SEQ ID NO: 112 - RNA, 21 nt, Artificial Sequence
  Synthetic
guggguccauccagucucuau

SEQ ID NO: 113 - RNA, 21 nt, Artificial Sequence
  Synthetic
uagaugguuccaagaagggag

SEQ ID NO: 114 - RNA, 21 nt, Artificial Sequence
  Synthetic
ucucacccagcacuucauaag

SEQ ID NO: 115 - RNA, 21 nt, Artificial Sequence
  Synthetic
agauuccccugccguugaagg

SEQ ID NO: 116 - RNA, 21 nt, Artificial Sequence
  Synthetic
ucaauugcuggaggugugggc

SEQ ID NO: 117 - RNA, 21 nt, Artificial Sequence
  Synthetic
acucagcuuaugaagugcugg

SEQ ID NO: 118 - RNA, 21 nt, Artificial Sequence
  Synthetic
gugcugggugagaccacugcc

SEQ ID NO: 119 - RNA, 21 nt, Artificial Sequence
  Synthetic
uuuuaugggcguagcugaaga

SEQ ID NO: 120 - RNA, 21 nt, Artificial Sequence
  Synthetic
ucgggaggagcagaggaggag

SEQ ID NO: 121 - DNA, 42 bp, Artificial Sequence
  Synthetic
agcaagtgagaagcatcgtgtcaaaatctcgccaacaagttg

SEQ ID NO: 122 - DNA, 22 bp, Artificial Sequence
  Synthetic
agcaagtgagaagcatcgtgtc

SEQ ID NO: 123 - DNA, 49 bp, Artificial Sequence
  Synthetic
aatgatacggcgaccaccgagatctacactctttccctacacgacgctc

SEQ ID NO: 124 - DNA, 88 bp, Artificial Sequence
  Synthetic
  misc_feature (25)..(34): n is a, c, g, or t
caagcagaagacggcatacgagatnnnnnnnnnngtctcgtgggctcggagatgtgtataagagacagtgtttccagagtactaaaac

SEQ ID NO: 125 - DNA, 83 bp, Artificial Sequence
  Synthetic
gttttagtactctggaaacagaatctactaaaacaaggcaaaatgccgtgtttatctcgtcaacttgttggcgagattttttt

SEQ ID NO: 126 - RNA, 83 nt, Artificial Sequence
  Synthetic
guuuuaguacucuggaaacagaaucuacuaaaacaaggcaaaaugccguguuuaucucgucaacuuguuggcgagauuuuuuu

(19) United States
(12) Patent Application Publication
(10) Pub. No.: US 2024/0026352 A1
(43) Pub. Date: Jan. 25, 2024

(54) TARGETED GENE REGULATION OF HUMAN IMMUNE CELLS WITH CRISPR-CAS SYSTEMS

(71) Applicant: Duke University, Durham, NC (US)

(72) Inventors: Charles A. Gersbach, Chapel Hill, NC (US); Sean McCutcheon, Durham, NC (US)

(21) Appl. No.: 18/036,862

(22) PCT Filed: Nov. 12, 2021
(86) PCT No.: PCT/US2021/059270

§ 371 Date: May 12, 2023

Related U.S. Application Data
(60) Provisional application No. 63/113,785, filed on Nov. 13, 2020.
(60) Provisional application No. 63/136,953, filed on Jan. 13, 2021.

(51) Int. Cl. C12N 15/11; C12N 9/22; C07K 14/47; C12N 15/90

(52) U.S. Cl. CPC C12N 15/11; C12N 9/22; C07K 14/4702; C12N 15/907; C12N 2310/20; C12N 2800/80; C07K 2319/80

(57) ABSTRACT

Disclosed herein are CRISPR/Cas systems comprising a fusion protein and at least one gRNA targeting a gene or a regulatory element thereof in a cell such as an immune cell, and vector compositions encoding the same. The systems and compositions may be used in methods of modulating expression of a gene in a cell such as an immune cell, as well as in methods of treating a disease such as cancer, autoimmune diseases, or viral infections.

### CROSS-REFERENCE TO RELATED APPLICATIONS

This application claims priority to U.S. Provisional Patent Application No. 63/113,785 filed Nov. 13, 2020, and U.S. Provisional Patent Application No. 63/136,953 filed Jan. 13, 2021, each of which is incorporated herein by reference in its entirety.

### FIELD

This disclosure relates to compositions and methods for programming immune cell function through targeted gene regulation and modulating expression of genes in immune cells.

### INTRODUCTION

Immunotherapy and regenerative medicine provide the exciting potential for cell-based therapies to treat many diseases and restore damaged tissues, but the inability to precisely control cell function has limited the ultimate success of this field. For over 40 years, gene therapy has been proposed as an approach to cure genetic diseases by adding functional copies of genes to the cells of patients with defined genetic mutations. However, this field has been limited by the available technologies for adding extra genetic material to human genomes. In recent years, the advent of synthetic biology has led to the development of technologies for precisely controlling gene networks that determine cell behavior. Several new technologies have emerged for manipulating genes in their native genomic context by engineering synthetic transcription factors that can be targeted to any DNA sequence. This includes new technologies that have enabled targeted human gene activation and repression, including the engineering of transcription factors based on zinc finger proteins, TALEs, and the CRISPR/Cas9 system. In addition, Adoptive T Cell Therapy (ACT) has revolutionized cancer treatment (FIG. 1), although ACT still faces several obstacles, such as impaired T cell trafficking, tumor heterogeneity, impaired T cell function, and poor T cell expansion and persistence. Genome and epigenome editing technologies may help overcome some of these challenges. Previous studies have demonstrated that CRISPR-Cas systems can successfully edit endogenous DNA sequences in T cells. However, these studies were largely limited to mutagenic, ‘loss-of-function’ perturbations with Cas9. Furthermore, CRISPR-based screens in primary human T cells, which can only be cultured ex vivo for limited time spans, has been hampered by low lentiviral transduction rates with Cas9-encoding vectors. There remains a need for the ability to precisely regulate any gene as it occurs naturally in the genome, such as the rewiring of genetic circuits to influence immune cell function, as a means to address a variety of diseases and disorders while circumventing some of the traditional challenges of gene therapy.

### SUMMARY

In an aspect, the disclosure relates to a CRISPR/Cas system including a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, and/or DNA demethylase activity; and at least one guide RNA (gRNA) targeting a gene or a regulatory element thereof in an immune cell.

In an aspect, the disclosure relates to a CRISPR/Cas system including a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, and/or DNA demethylase activity; and at least one guide RNA (gRNA) targeting a gene selected from B2M, TIGIT, CD2, EGFR, and IL2RA, or a regulatory element thereof in a cell. In some embodiments, the cell is an immune cell.

In some embodiments, the immune cell is a T cell. In some embodiments, the first polypeptide domain comprises a Cas9 protein. In some embodiments, the first polypeptide domain comprises a Staphylococcus aureus Cas9 protein (SaCas9). In some embodiments, the first polypeptide domain comprises a nuclease-inactivated Cas9 protein (dCas9). In some embodiments, the first polypeptide domain comprises a nuclease-inactivated Staphylococcus aureus Cas9 protein (dSaCas9). In some embodiments, the gRNA targets a gene selected from B2M, TIGIT, CD2, EGFR, and IL2RA, or a regulatory element thereof. In some embodiments, the gRNA comprises a polynucleotide sequence selected from SEQ ID NOs: 58-70 or 102-120, a complement thereof, a variant thereof, or a fragment thereof, or is encoded by or targets a polynucleotide sequence selected from SEQ ID NOs: 45-57 or 83-101. In some embodiments, the gRNA targets B2M or a regulatory element thereof. In some embodiments, the gRNA targets a sequence within 500 base pairs of the transcriptional start site of B2M. In some embodiments, the gRNA comprises a polynucleotide sequence selected from SEQ ID NOs: 66-70, a complement thereof, a variant thereof, or a fragment thereof. In some embodiments, the gRNA is encoded by or targets a polynucleotide comprising a sequence selected from SEQ ID NOs: 53-57. In some embodiments, the gRNA targets TIGIT or a regulatory element thereof. In some embodiments, the gRNA targets a sequence within 500 base pairs of the transcriptional start site of TIGIT. In some embodiments, the gRNA comprises the polynucleotide sequence of SEQ ID NO: 110, a complement thereof, a variant thereof, or a fragment thereof. In some embodiments, the gRNA is encoded by or targets a polynucleotide comprising the sequence of SEQ ID NO: 91. In some embodiments, the gRNA targets CD2 or a regulatory element thereof. In some embodiments, the gRNA targets a sequence within 500 base pairs of the transcriptional start site of CD2. In some embodiments, the gRNA comprises a polynucleotide sequence selected from SEQ ID NOs: 58-65 or 102-109, a complement thereof, a variant thereof, or a fragment thereof. In some embodiments, the gRNA is encoded by or targets a polynucleotide comprising a sequence selected from SEQ ID NOs: 45-52 or 83-90. In some embodiments, the gRNA targets EGFR or a regulatory element thereof. In some embodiments, the gRNA targets a sequence within 500 base pairs of the transcriptional start site of EGFR. In some embodiments, the gRNA comprises the polynucleotide sequence of SEQ ID NO: 101, a complement thereof, a variant thereof, or a fragment thereof. In some embodiments, the gRNA is encoded by or targets a polynucleotide comprising the sequence of SEQ ID NO: 120. In some embodiments, the gRNA targets IL2RA or a regulatory element thereof. In some embodiments, the gRNA targets a sequence within 500 base pairs of the transcriptional start site of IL2RA. In some embodiments, the gRNA comprises a polynucleotide sequence selected from SEQ ID NOs: 111-119, a complement thereof, a variant thereof, or a fragment thereof. In some embodiments, the gRNA is encoded by or targets a polynucleotide comprising a sequence selected from SEQ ID NOs: 92-100. In some embodiments, the gRNA further comprises the polynucleotide sequence of SEQ ID NO: 19 or 126. In some embodiments, the second polypeptide domain has transcription repression activity. In some embodiments, the at least one guide RNA (gRNA) targets a gene selected from B2M, TIGIT, and CD2, or a regulatory element thereof. In some embodiments, the second polypeptide domain comprises a KRAB domain, EED domain, MECP2 domain, ERF repressor domain, Mxi1 repressor domain, SID4X repressor domain, Mad-SID repressor domain, DNMT3A or DNMT3L or fusion thereof, LSD1 histone demethylase, or TATA box binding protein domain. In some embodiments, the fusion protein comprises dSaCas9-KRAB. In some embodiments, the second polypeptide domain has transcription activation activity. In some embodiments, the at least one guide RNA (gRNA) targets a gene selected from CD2, EGFR, and IL2RA, or a regulatory element thereof. In some embodiments, the second polypeptide domain comprises a VP16, a VP48, a VP64, a p65, a TET1, a VPR, a VPH, a Rta, or a p300 protein, or a fragment thereof or a combination thereof. In some embodiments, the fusion protein comprises dSaCas9-VP64, VP64-dSaCas9-VP64, or dSaCas9-p300^{core}.

In a further aspect, the disclosure relates to an isolated polynucleotide encoding a CRISPR/Cas system as detailed herein.

In a further aspect, the disclosure relates to a vector comprising an isolated polynucleotide as detailed herein.

In a further aspect, the disclosure relates to a cell comprising an isolated polynucleotide as detailed herein or a vector as detailed herein.

In a further aspect, the disclosure relates to a vector composition including a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and a polynucleotide sequence encoding at least one guide RNA (gRNA) targeting a gene or a regulatory element thereof in an immune cell.

In a further aspect, the disclosure relates to a vector composition including a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and a polynucleotide sequence encoding at least one guide RNA (gRNA) targeting a gene selected from B2M, TIGIT, CD2, EGFR, and IL2RA, or a regulatory element thereof in a cell. In some embodiments, the cell is an immune cell.

In some embodiments, the immune cell is a T cell. In some embodiments, the first polypeptide domain comprises a Cas9 protein. In some embodiments, the first polypeptide domain comprises a Staphylococcus aureus Cas9 protein (SaCas9). In some embodiments, the first polypeptide domain comprises a nuclease-inactivated Cas9 protein (dCas9). In some embodiments, the first polypeptide domain comprises a nuclease-inactivated Staphylococcus aureus Cas9 protein (dSaCas9). In some embodiments, the vector composition comprises a first vector comprising the polynucleotide sequence encoding a fusion protein, and a second vector comprising the polynucleotide sequence encoding at least one gRNA. In some embodiments, the vector composition comprises a single vector comprising the polynucleotide sequence encoding a fusion protein and the polynucleotide sequence encoding the at least one gRNA. In some embodiments, the vector composition further includes a polynucleotide sequence encoding a reporter protein operably linked to the polynucleotide sequence encoding the fusion protein. In some embodiments, the reporter protein comprises a fluorescent protein and/or a protein detectable with an antibody. In some embodiments, the vector composition further includes a polynucleotide sequence encoding a 2A self-cleaving peptide operably linked to the polynucleotide sequence encoding the fusion protein and to the polynucleotide sequence encoding the reporter protein, wherein the T2A polynucleotide sequence is between the polynucleotide sequence encoding the fusion protein and the polynucleotide sequence encoding the reporter protein. In some embodiments, the gRNA targets a gene selected from B2M, TIGIT, CD2, EGFR, and IL2RA, or a regulatory element thereof. In some embodiments, the gRNA comprises a polynucleotide sequence selected from SEQ ID NOs: 58-70 or 102-120, a complement thereof, a variant thereof, or a fragment thereof, or is encoded by or targets a polynucleotide sequence selected from SEQ ID NOs: 45-57 or 83-101. In some embodiments, the gRNA targets B2M or a regulatory element thereof. In some embodiments, the gRNA targets a sequence within 500 base pairs of the transcriptional start site of B2M. In some embodiments, the gRNA comprises a polynucleotide sequence selected from SEQ ID NOs: 66-70, a complement thereof, a variant thereof, or a fragment thereof. In some embodiments, the gRNA is encoded by or targets a polynucleotide sequence selected from SEQ ID NOs: 53-57. In some embodiments, the gRNA targets TIGIT or a regulatory element thereof. In some embodiments, the gRNA targets a sequence within 500 base pairs of the transcriptional start site of TIGIT. In some embodiments, the gRNA comprises the polynucleotide sequence of SEQ ID NO: 110, a complement thereof, a variant thereof, or a fragment thereof. In some embodiments, the gRNA is encoded by or targets a polynucleotide comprising the sequence of SEQ ID NO: 91. In some embodiments, the gRNA targets CD2 or a regulatory element thereof. In some embodiments, the gRNA targets a sequence within 500 base pairs of the transcriptional start site of CD2. In some embodiments, the gRNA comprises a polynucleotide sequence selected from SEQ ID NOs: 58-65 or 102-109, a complement thereof, a variant thereof, or a fragment thereof. In some embodiments, the gRNA is encoded by or targets a polynucleotide comprising a sequence selected from SEQ ID NOs: 45-52 or 83-90. In some embodiments, the gRNA targets EGFR or a regulatory element thereof. In some embodiments, the gRNA targets a sequence within 500 base pairs of the transcriptional start site of EGFR. In some embodiments, the gRNA comprises the polynucleotide sequence of SEQ ID NO: 120, a complement thereof, a variant thereof, or a fragment thereof. In some embodiments, the gRNA is encoded by or targets a polynucleotide comprising the sequence of SEQ ID NO: 101. In some embodiments, the gRNA targets IL2RA or a regulatory element thereof. In some embodiments, the gRNA targets a sequence within 500 base pairs of the transcriptional start site of IL2RA. In some embodiments, the gRNA comprises a polynucleotide sequence selected from SEQ ID NOs: 111-119, a complement thereof, a variant thereof, or a fragment thereof. In some embodiments, the gRNA is encoded by or targets a polynucleotide sequence selected from SEQ ID NOs: 92-100. In some embodiments, the gRNA further comprises the polynucleotide sequence of SEQ ID NO: 19 or 126. In some embodiments, the second polypeptide domain has transcription repression activity. In some embodiments, the at least one guide RNA (gRNA) targets a gene selected from B2M, TIGIT, and CD2, or a regulatory element thereof. In some embodiments, the second polypeptide domain comprises a KRAB domain, EED domain, MECP2 domain, DNMT3A or DNMT3L or fusion thereof, ERF repressor domain, Mxi1 repressor domain, SID4X repressor domain, Mad-SID repressor domain, LSD1 histone demethylase, or TATA box binding protein domain. In some embodiments, the fusion protein comprises dSaCas9-KRAB. In some embodiments, the second polypeptide domain has transcription activation activity. In some embodiments, the at least one guide RNA (gRNA) targets a gene selected from CD2, EGFR, and IL2RA, or a regulatory element thereof. In some embodiments, the second polypeptide domain comprises a VP16, a VP48, a VP64, a p65, a TET1, a VPR, a VPH, a Rta, or a p300 protein, or a fragment thereof or a combination thereof. In some embodiments, the fusion protein comprises dSaCas9-VP64, VP64-dSaCas9-VP64, or dSaCas9-p300^{core}. In some embodiments, the vector composition further includes a human Pol III U6 promoter upstream of and driving expression of the polynucleotide sequence encoding the gRNA, wherein the human Pol III U6 promoter and the polynucleotide sequence encoding the gRNA are orientated in the opposite direction from the polynucleotide sequence encoding the fusion protein. In some embodiments, the vector composition comprises a lentiviral vector comprising the polynucleotide sequence encoding a fusion protein and/or the polynucleotide sequence encoding the gRNA.

Another aspect of the disclosure provides a method of modulating expression of a gene in a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of reducing B2M expression in a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of reducing immunological activity of a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of reducing TIGIT expression in a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of increasing an immune cell's ability to kill a cancer cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of reducing CD2 expression in a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of increasing CD2 expression in a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of increasing EGFR expression in a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a method of increasing IL2RA expression in a cell. The method may include administering to the cell a CRISPR/Cas system as detailed herein, an isolated polynucleotide as detailed herein, a vector as detailed herein, or a vector composition as detailed herein. In some embodiments, the cell is an immune cell. In some embodiments, the immune cell is a T cell.

Another aspect of the disclosure provides a cell modified by a method as detailed herein.

Another aspect of the disclosure provides a method of treating a subject having a disease. The method may include administering to the subject a CRISPR/Cas system as detailed herein, an isolated polynucleotide as detailed herein, a vector as detailed herein, a cell as detailed herein, or a vector composition as detailed herein. In some embodiments, the disease comprises cancer, an autoimmune disease, or a viral infection.

Another aspect of the disclosure provides a method of screening for one or more putative gene regulatory elements in a genome that modulate a gene target or a phenotype of an immune cell. The method may include (a) contacting a plurality of modified target immune cells with a library of gRNAs, each gRNA targeting a gene regulatory element in an immune cell, thereby generating a pool of test immune cells, (b) selecting a population of test immune cells having a modulated gene or phenotype; (c) quantifying the frequency of the gRNAs within the population of selected immune cells, wherein the gRNAs that target gene regulatory elements that modulate the phenotype are overrepresented or underrepresented in the selected immune cells; and (d) identifying and characterizing the gRNAs within the population of selected immune cells thereby identifying the gene regulatory elements that modulate the phenotype, wherein the modified target immune cell comprises a fusion protein, the fusion protein comprising a first polypeptide domain comprising a Cas protein and a second polypeptide domain having an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity. In some embodiments, the immune cell is a T cell. In some embodiments, the first polypeptide domain comprises a Cas9 protein. In some embodiments, the first polypeptide domain comprises a Staphylococcus aureus Cas9 protein (SaCas9). In some embodiments, the first polypeptide domain comprises a nuclease-inactivated Staphylococcus aureus Cas9 protein (dSaCas9).

Another aspect of the disclosure provides method of screening a library of gRNAs for modulation of gene expression in a cell. The method may include (a) generating a library of vectors with a library of gRNAs, each gRNA targeting a target gene or a regulatory element thereof in a cell, the library of vectors comprising a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; a polynucleotide sequence encoding a reporter protein operably linked to the polynucleotide sequence encoding the fusion protein; and a polynucleotide sequence encoding one of the gRNAs; (b) transducing a plurality of cells with the library of gRNAs; (c) culturing the transduced cells; (d) sorting the cultured cells based on the growth of the cells or on the level of expression of the gene or the reporter protein; and (e) sequencing the gRNA from each cell sorted in step (d). In some embodiments, the reporter protein comprises a fluorescent protein and/or a protein detectable with an antibody, and wherein the cultured cells are sorted in step (d) based on the level of expression of the reporter protein. In some embodiments, the cell is an immune cell. In some embodiments, the immune cell is a T cell. In some embodiments, the first polypeptide domain comprises a Cas9 protein. In some embodiments, the first polypeptide domain comprises a Staphylococcus aureus Cas9 protein (SaCas9). In some embodiments, the first polypeptide domain comprises a nuclease-inactivated Staphylococcus aureus Cas9 protein (dSaCas9). In some embodiments, the library of vectors further comprises a polynucleotide sequence encoding a 2A self-cleaving peptide operably linked to the polynucleotide sequence encoding the fusion protein and to the polynucleotide sequence encoding the reporter protein, wherein the polynucleotide sequence encoding a 2A self-cleaving peptide is between the polynucleotide sequence encoding the fusion protein and the polynucleotide sequence encoding the reporter protein. In some embodiments, the method further includes (f) identifying the target gene of the gRNA sequenced in step (e). In some embodiments, the method further includes (g) modulating the level of the gene target discovered in (f) or modulating the activity of the protein produced from the gene target discovered in (f) for enhancing properties of a cell therapy.

Another aspect of the disclosure provides a method of screening a library of gRNAs for modulation of gene expression in a cell. The method may include (a) generating a library of vectors with a library of gRNAs, each gRNA targeting a target gene or a regulatory element thereof in a cell, the library of vectors comprising a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and a polynucleotide sequence encoding one of the gRNAs; (b) transducing a plurality of cells with the library of gRNAs; (c) culturing the transduced cells; (d) capturing the gRNA from the transduced cells; and (e) sequencing the gRNA from each transduced cell captured in step (d). In some embodiments, the gRNA from the transduced cells is captured with single cell technology in step (d). In some embodiments, the method further includes determining the level of mRNA expression and/or the level of protein expression in the transduced cells. In some embodiments, the method further includes grouping transduced cells having the same gRNA; and comparing the target gene expression of transduced cells having the same gRNA, at the mRNA and/or protein level, to the target gene expression of cells without the same gRNA. In some embodiments, the method further includes identifying the target gene of the gRNA sequenced in step (e). In some embodiments, the method further includes modulating the level of the gene target or modulating the activity of the protein produced from the gene target for enhancing properties of a cell therapy. In some embodiments, the cell is an immune cell. In some embodiments, the immune cell is a T cell. In some embodiments, the first polypeptide domain comprises a Staphylococcus aureus Cas9 protein (SaCas9). In some embodiments, the first polypeptide domain comprises a nuclease-inactivated Staphylococcus aureus Cas9 protein (dSaCas9).

The disclosure provides for other aspects and embodiments that will be apparent in light of the following detailed description and accompanying figures.

### BRIEF DESCRIPTION OF THE DRAWINGS

FIG. 1 is a schematic diagram of the steps of Adoptive T Cell Therapy (ACT) for cancer treatment.

FIG. 2A is a schematic diagram of the Lentiviral (LV) dSaCas9 Vector (CRISPRi construct). The human ubiquitin promoter drives expression of dSaCas9 fused to a KRAB repressive domain, which is linked to GFP expression by a 2A self-cleaving peptide sequence. FIG. 2B are representative scatter plots of T cells that were either untreated or transduced with dSaCas9 LV and assayed for GFP expression on day 4 after transduction. FIG. 2C is a graph showing summary statistics of the GFP+ cells (%) for untreated and transduced cells. An asterisk denotes P<0.05.

FIG. 3A is a schematic diagram of the Lentiviral All-in-One SaCas9 Vector. The human ubiquitin promoter drives expression of dSaCas9 fused to a KRAB repressive domain, which is linked to GFP expression by a 2A self-cleaving peptide sequence. A human Pol III U6 promoter orientated in the opposite direction drives expression of the single gRNA. FIG. 3B is a schematic diagram of the screening pipeline.

FIG. 4A is a volcano plot (−log 10 of adjusted P values vs fold-change in counts between high and low bins) of gRNAs for the CRISPRi CD2 screen. Non-targeting gRNAs are labeled in light gray, targeting gRNAs are in dark gray (with statistically significant gRNAs in open circles). FIG. 4B is a graph showing fold change vs. gRNA positioning relative to the TSS.

FIG. 5A are representative density plots of CD2 repression for each gRNA. The CD2-low gate was set with non-targeting control gRNA. FIG. 5B is a graph showing the percentage of CD2+ cells for each gRNA.

FIG. 6A is a graph showing gRNA activity is correlated with log 2(fc) from the screen. The graph compares CD2 protein levels (MRI=mean fluorescence intensity) on the y-axis for each gRNA to the strength of depletion in the screen for that gRNA. On the x-axis, log 2(fc) is log 2(fold-change), wherein “fold-change” is the difference in gRNA abundance in the screen between the CD2 high and CD2 low populations. FIG. 6B is a graph showing the fold change in CD2 mRNA for each gRNA, as determine by RT-qPCR.

FIG. 7A is a schematic diagram for the design of B2M gRNAs, with the UCSC genome browser track with upstream and downstream most gRNAs targeting B2M annotated. DNase-seq and ChIP-seq tracks of histone marks associated with active transcription and open chromatin are also displayed. FIG. 7B is a histogram of B2M gRNA abundance relative to the TSS.

FIG. 8A is a graph showing B2M distribution for unstained and stained T cells. FIG. 8B is a graph showing the lower and upper ˜10% tails of B2M-expressing cells that were sorted off into low and high bins. FIG. 8C is a volcano plot (−log 10 of adjusted P values vs fold-change in counts between high and low bins) of gRNAs for the CRISPRi B2M screen. Non-targeting gRNAs are labeled in light gray, targeting gRNAs are in dark gray.

FIG. 9 is a graph showing the statistical significance vs. gRNA positioning relative to the TSS for the CRISPRi B2M screen. White circles denote statistically significant gRNAs (P_{adjusted}<0.05). The top 5 gRNA hits (labeled H1-H5) were cloned for individual validation.

FIG. 10A are representative density plots of B2M repression for non-targeting (NT), H1, H2, and H4 across 3 time points (day 3, 6, and 9). The B2M low gate was set with the non-targeting control. gRNAs differed markedly in their kinetics of repression. FIG. 10B is a scatter plot of B2M repression over time for each gRNA. Each solid point/line represents the averaged percentage of silenced B2M cells across 3 replicates (with individual replicates being plotted opaque.

FIG. 11A is a graph showing the summary statistics of the percentage of cells repressing B2M across replicates for each gRNA. FIG. 11B is a graph of the results of RT-qPCR of B2M within transduced cells.

FIG. 12 is a schematic diagram of the CD2 multimodal scRNA-seq screen.

FIG. 13A-FIG. 13B are graphs showing the level of CD2 gene expression at the protein (FIG. 13A) and the mRNA (FIG. 13B) level.

FIG. 14 shows the greatest effect on CD2 expression was observed for gRNA7, gRNA8, gRNA9, gRNA10, gRNA11, gRNA12, gRNA15, and gRNA16.

FIG. 15A-15C are graphs showing multimodal CD2 repression at the single-cell level.

FIG. 16A-16B are graphs of CD2 gene expression with different gRNAs.

FIG. 17 is a schematic diagram of the method used to isolate T cells from healthy and diseased lung tissue.

FIG. 18A are results from FACS, and FIG. 18B is the corresponding graph, showing robust TIGIT repression in TILs.

FIG. 19 are results from FACS showing that repression of TIGIT expression was dependent on SaCas9 and the targeting gRNA.

FIG. 20 are results from FACS showing B2M repression in TILs that had been expanded in high concentrations of IL-2 for 2-3 weeks prior to transduction.

FIG. 21 is a schematic diagram of the CRISPRa screen.

FIG. 22A-22C are graphs showing gene expression with various gRNAs compared between dSaCas9-VP64 and VP64-dSaCas9-VP64 fusion proteins.

FIG. 23 is a graph showing the placement of the gRNAs, showing that they predominantly fell near the TSS and target strict PAM.

FIG. 24A and FIG. 24B are graphs showing IL2RA gene expression with various gRNAs compared between dSaCas9-VP64 and VP64-dSaCas9-VP64 fusion proteins. FIG. 24C is a graph comparing mRNA levels with dSaCas9-VP64, VP64-dSaCas9-VP64, or VP64-dSpCas9-VP64 fusion proteins. FIG. 24D is graph from FACS showing that VP64dSaCas9VP64 can upregulate endogenous genes such as EGFR in primary human T cells.

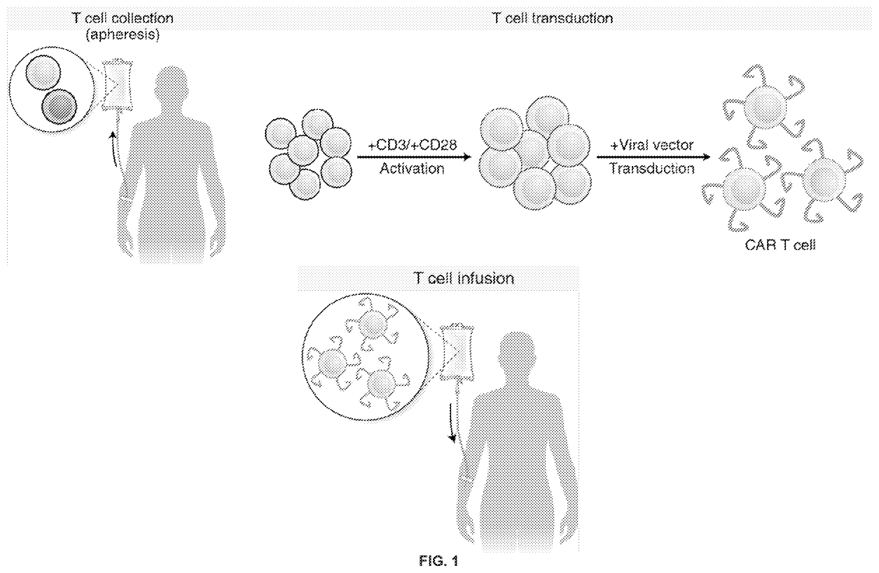

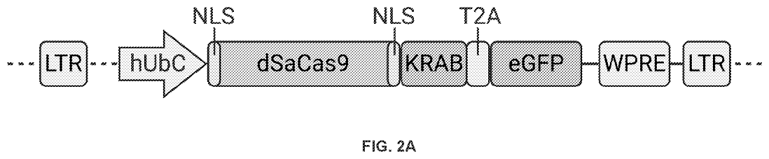

### DETAILED DESCRIPTION

Described herein are compositions and methods for modulating expression of genes in cells, such as immune cells like T cells, with CRISPR/Cas systems, as well as methods of screening potential gRNAs for modulating expression of genes in cells. Epigenome editing in human primary immune cells has previously been elusive due to low transduction rates and poor expression of CRISPR-Cas effectors and the limited culture duration of primary cells. Detailed herein is a CRISPR-based platform that may be used to regulate gene expression and rapidly identify optimal single gRNAs in immune cells such as human primary T cells.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Allogeneic” refers to any material derived from another subject of the same species. Allogeneic cells are genetically distinct and immunologically incompatible yet belong to the same species. Typically, “allogeneic” is used to define cells, such as stem cells, that are transplanted from a donor to a recipient of the same species. “Allogeneic” may also be used to define T cells. “Allotransplant” refers to the transplantation of cells, tissues, or organs to a recipient from a genetically non-identical donor of the same species.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

An “autoimmune disease” is a condition arising from an abnormal immune response to a functioning body part. Autoimmune diseases may be divided into two general types, namely systemic autoimmune diseases (exemplified by arthritis, lupus, and scleroderma), and organ0specific (exemplified by multiple sclerosis, diabetes and atherosclerosis, in which latter case the vasculature is regarded as a specific organ). Autoimmune diseases include, for example, rheumatoid arthritis, graft versus host disease, myasthenia gravis, systemic lupus erythromatosis (SLE), scleroderma, multiple sclerosis, diabetes, organ rejection, inflammatory bowel disease, autoimmune thyroiditis, autoimmune uveoretinitis, and psoriasis.

“Autologous” refers to any material derived from a subject and re-introduced to the same subject.

“Binding region” as used herein refers to the region within a target region that is recognized and bound by the CRISPR/Cas-based gene editing system.

“Cancer” refers to a neoplasm or tumor resulting from abnormal and uncontrolled growth of cells. Cancer may also be referred to as a cellular-proliferative disease. Cancer may include different histological types, cell types, and different stages of cancer, such as, for example, primary tumor or metastatic growth. Cancer may include, for example, breast cancer, cholangiocellular carcinoma, colorectal cancer, endometriosis, esophageal cancer, gastric cancer, diffused type gastric cancer, pancreatic cancer, renal carcinoma, soft tissue tumor, testicular cancer, cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma, non-small cell lung cancer (NSCLC). small cell lung cancer (SCLC); Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defomians), meninges (meningioma, meningiosarcoma, gliomatosis), brain (astrocytoma, medulloblastoma, glioma, ependymoma, germinoma [pinealoma], glioblastoma, glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian cancer, ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma], CML; Skin: melanoma, malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. In some embodiments, the cancer comprises at least one of breast cancer, ovarian cancer, lung cancer such as non-small cell lung cancer (NSCLC), pancreatic cancer, stomach cancer, colorectal cancer, prostate cancer, uterine cancer, bladder cancer, and liver cancer.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be an subject or cell without an agonist as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Immune cells” refer to cells of the immune system, which defend the body against disease and foreign materials. Non-limiting examples of immune cells include dendritic cells, such as bone marrow-derived dendritic cells; lymphocytes, such as B cells, T cells, and natural killer cells; and macrophages. The immune cells may, in some embodiments, be derived from bone marrow, spleen, or blood from a suitable subject.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic. DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is a gene expressed differentially in an immune cell, such as a T cell.

“Target region” as used herein refers to the region of the target gene to which the CRISPR/Cas9-based gene editing or targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or“heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. DNA TARGETING SYSTEMS

A “DNA Targeting System” as used herein is a system capable of specifically targeting a particular region of DNA and activating gene expression by binding to that region. Non-limiting examples of these systems are CRISPR-Cas-based systems, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

Each of these systems comprises a DNA-binding portion or domain, such as a guide RNA, a ZF, or a TALE, that specifically recognizes and binds to a particular target region of a target DNA. The DNA-binding portion (for example, Cas protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity, to form a fusion protein. Exemplary second polypeptide domains are detailed further below (see “Cas Fusion Protein). For example, the DNA-binding portion can be linked to an activator and thus guide the activator to a specific target region of the target DNA. Similarly, the DNA-binding portion can be linked to a repressor and thus guide the repressor to a specific target region of the target DNA.

In some embodiments, the DNA-binding portion comprises a Cas protein, such as a Cas9 protein. Some CRISPR-Cas-based systems can operate to activate or repress expression using the Cas protein alone, not linked to an activator or repressor. For example, a nuclease-null Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor. Some CRISPR-Cas-based systems can operate to activate or repress expression using the Cas protein linked to a second protein domain, such as, for example, an activator or repressor.

### 3. CRISPR/CAS-BASED GENE EDITING SYSTEM

Provided herein are CRISPR/Cas-based gene editing systems. The CRISPR/Cas-based gene editing system may be used to modulate expression of a target gene in a cell, such as an immune cell. The CRISPR/Cas-based gene editing system may include a Cas protein or a fusion protein, and at least one gRNA, and may also be referred to as a “CRISPR-Cas system.”

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, and Cascade proteins. Cas12a may also be referred to as “Cpf1.” Cas12a causes a staggered cut in double stranded DNA, while Cas9 produces a blunt cut. In some embodiments, the Cas protein comprises Cas12a. In some embodiments, the Cas protein comprises Cas9. Cas9 forms a complex with the 3′ end of the gRNA, and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of Streptococcus pyogenes was shown to function in human cells for genome engineering. In this system, the Cas9 protein was directed to genomic target sites by a synthetically reconstituted “guide RNA” (“gRNA”), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, cell growth, or wound healing. The CRISPR/Cas9-based gene editing system can include a Cas9 protein or a Cas9 fusion protein.

a. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus, Acidovorax avenae, Actinobacillus pleuropneumoniae, Actinobacillus succinogenes, Actinobacillus suis, Actinomyces sp., cycliphilus denitrifcans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebactenum diphtheria, Corynebacterium matruchotii, Dinoroseobacter shibae, Eubacterium dolichum, Gamma proteobacterium, Gluconacetobacter diazotrophicus, Haemophilus parainfluenzae. Haemophilus sputorum, Helicobacter canadensis, Helicobacter cinaedi, Helicobacter mustelae, Ilyobacter polytropus, Kingella kingae, Lactobacillus cispatus, Listeria ivanovii, Listeria monocytogenes, Listenaceae bacterium, Methylocystis sp., Methylosinus trichosporium, Mobiluncus mulieris, Neisseria bacilliformis, Neisseria cinerea, Neissena flavescens, Neissena lactamica, Neisseria sp., Neisseria wadsworthii, Nitrosomonas sp., Parvibaculum lavamentivorans, Pasteurella multocida, Phascolarctobacterium succinatutens, Ralstonia syzygi, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 20. In certain embodiments, the Cas9 molecule is a Staphylococcus aureus Cas9 molecule (also referred herein as “SaCas9”). SaCas9 may comprise an amino acid sequence of SEQ ID NO: 21.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The targeting sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G; V=A or C or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 73).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A. N854A, N863A and/or D986A. A S. pyogenes Cas9 protein with the D10A mutation may comprise an amino acid sequence of SEQ ID NO: 22. A S. pyogenes Cas9 protein with D10A and H849A mutations may comprise an amino acid sequence of SEQ ID NO: 23. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 24. In certain embodiments, the mutant S. aureus Cas9 molecule comprises a N580A mutation. The nucleotide sequence encoding this mutant S. aureus Cas9 molecule is set forth in SEQ ID NO: 25.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 26. Exemplary codon optimized nucleic acid sequences encoding a Cas9 molecule of S. aureus, and optionally containing nuclear localization sequences (NLSs), are set forth in SEQ ID NOs: 27-33. Another exemplary codon optimized nucleic acid sequence encoding a Cas9 molecule of S. aureus comprises the nucleotides 1293-4451 of SEQ ID NO: 34.

b. Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain has a different activity that what is endogenous to Cas protein. For example, the second polypeptide domain may have an activity such as transcription activation activity, transcription repression activity, transcription release factor activity, histone modification activity, nuclease activity, nucleic acid association activity, histone methylase activity. DNA methylase activity, histone demethylase activity, DNA demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)ₙ, wherein n is an integer between 0 and 10 (SEQ ID NO: 74). In a GS linker, n can be adjusted to optimize the linker length and achieve appropriate separation of the functional domains. Other examples of linkers may include, for example, Gly-Gly-Gly-Gly-Gly (SEQ ID NO: 75), Gly-Gly-Ala-Gly-Gly (SEQ ID NO: 76), Gly/Ser rich linkers such as Gly-Gly-Gly-Gly-Ser-Ser-Ser (SEQ ID NO: 77), or Gly/Ala rich linkers such as Gly-Gly-Gly-Gly-Ala-Ala-Ala (SEQ ID NO: 78).

i) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, or p300, or a combination thereof, or a partially or fully functional fragment thereof. For example, the fusion protein may comprise dCas9-p300. In some embodiments, p300 comprises a polypeptide having the amino acid sequence of SEQ ID NO: 35 or SEQ ID NO: 36. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 37, encoded by the polynucleotide of SEQ ID NO: 38. VPH may comprise a polypeptide having the amino acid sequence of SEQ ID NO: 79, encoded by the polynucleotide of SEQ ID NO: 80. VPR may comprise a polypeptide having the amino acid sequence of SEQ ID NO: 81, encoded by the polynucleotide of SEQ ID NO: 82.

ii) Transcription Repression Activity

The second polypeptide domain can have transcription repression activity. Non-limiting examples of repressors include Kruppel associated box activity such as a KRAB domain or KRAB, MECP2, EED, ERF repressor domain (ERD). Mad mSIN3 interaction domain (SID) or Mad-SID repressor domain, SID4X repressor domain, Mxi1 repressor domain. SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, Mxi1 repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the second polypeptide domain comprises KRAB. For example, the fusion protein may be S. pyogenes dCas9-KRAB (polynucleotide sequence SEQ ID NO: 39; protein sequence SEQ ID NO: 40). The fusion protein may be S. aureus dCas9-KRAB (polynucleotide sequence SEQ ID NO: 41; protein sequence SEQ ID NO: 42).

iii) Transcription Release Factor Activity

The second polypeptide domain can have transcription release factor activity. The second polypeptide domain can have eukaryotic release factor 1 (ERF1) activity or eukaryotic release factor 3 (ERF3) activity.

iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 35 or SEQ ID NO: 36.

v) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

vii) Methylase Activity

The second polypeptide domain can have methylase activity, which involves transferring a methyl group to DNA, RNA, protein, small molecule, cytosine, or adenine. In some embodiments, the second polypeptide domain has histone methylase activity. In some embodiments, the second polypeptide domain has DNA methylase activity. In some embodiments, the second polypeptide domain includes a DNA methyltransferase.

viii) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; polynucleotide sequence SEQ ID NO: 43; amino acid sequence SEQ ID NO: 44). In some embodiments, the second polypeptide domain has histone demethylase activity. In some embodiments, the second polypeptide domain has DNA demethylase activity.

c. Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA-tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 or 126 (RNA), which is encoded by a sequence comprising SEQ ID NO: 18 or 125 (DNA), respectively. The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target a region that affects gene transcription. The gRNA may target a region of a gene that is specific to or highly expressed in certain cells, such as immune cells. The gRNA may target a region of a gene selected from B2M, TIGIT, CD2, IL2RA, and EGFR, or a regulatory element thereof. The gRNA may bind and target or be encoded by a polynucleotide sequence comprising at least one of SEQ ID NOs: 45-57 or 83-90 or 91-101, or a complement thereof, or a variant thereof, or a truncation thereof. The gRNA may comprise a polynucleotide sequence selected from SEQ ID NOs: 58-70 or 102-120, or a complement thereof, or a variant thereof, or a truncation thereof. A truncation may be 1, 2, 3, 4, 5, 6, 7, 8, or 9 nucleotides shorter than the sequence of SEQ ID NOs: 45-70 or 83-120. Examples of gRNAs are shown in TABLE 1.

In some embodiments, the gRNA targets B2M or a regulatory element thereof. The gRNA targeting B2M may be used with a Cas9 fusion protein comprising a second polypeptide domain having transcription repression activity, thereby repressing or reducing expression of the B2M gene. B2M (also known as P2 microglobulin) may be part of the major histocompatibility complex (MHC). Repression or reduction of B2M expression in a cell may facilitate the administration of the cell to a different subject from which the cell was originally derived. Repression or reduction of B2M expression in a cell may facilitate the administration of the cell as an allogenic transplant. gRNAs targeting B2M may be used in combination with gRNAs targeting other genes. In some embodiments, the gRNA targets B2M or a regulatory element thereof and binds and targets a polynucleotide sequence comprising at least one of SEQ ID NOs: 45-52 or 83-90, or a complement thereof, or a variant thereof, or a truncation thereof. In some embodiments, the gRNA targets B2M or a regulatory element thereof and is encoded by a polynucleotide sequence comprising at least one of SEQ ID NOs: 45-52 or 83-90, or a complement thereof, or a variant thereof, or a truncation thereof. In some embodiments, the gRNA targets B2M or a regulatory element thereof and comprises a polynucleotide sequence comprising at least one of SEQ ID NOs: 58-85 or 102-109, or a complement thereof, or a variant thereof, or a truncation thereof.

In some embodiments, the gRNA targets TIGIT or a regulatory element thereof. The gRNA targeting TIGIT may be used with a Cas9 fusion protein comprising a second polypeptide domain having transcription repression activity, thereby repressing or reducing expression of the TIGIT gene. TIGIT (also known as T cell immunoreceptor with Ig and ITIM domains) is an immune receptor present on some T cells and natural killer cells (NK). TIGIT may be overexpressed on tumor antigen-specific (TA-specific) CD8+ T cells and/or CD8+ tumor infiltrating lymphocytes (TILs). TIGIT is also known as a checkpoint inhibitor. TIGIT expressed on T cells or TILs may signal the T cell or TIL to not kill a cancer cell. Repression or reduction of TIGIT expression in a cell, such as a T cell or TIL, may signal the T cell or TIL to kill a cancer cell. gRNAs targeting TIGIT may be used in combination with gRNAs targeting other genes. In some embodiments, the gRNA targets TIGIT or a regulatory element thereof and binds and targets a polynucleotide sequence comprising SEQ ID NO: 91, or a complement thereof, or a variant thereof, or a truncation thereof. In some embodiments, the gRNA targets TIGIT or a regulatory element thereof and is encoded by a polynucleotide sequence comprising SEQ ID NO: 91, or a complement thereof, or a variant thereof, or a truncation thereof. In some embodiments, the gRNA targets TIGIT or a regulatory element thereof and comprises a polynucleotide sequence comprising SEQ ID NO: 110, or a complement thereof, or a variant thereof, or a truncation thereof.

In some embodiments, the gRNA targets CD2 or a regulatory element thereof. gRNAs targeting CD2 may be used in combination with gRNAs targeting other genes. In some embodiments, the gRNA targets CD2 or a regulatory element thereof and binds and targets a polynucleotide sequence comprising at least one of SEQ ID NOs: 45-52 or 83-90, or a complement thereof, or a variant thereof, or a truncation thereof. In some embodiments, the gRNA targets CD2 or a regulatory element thereof and is encoded by a polynucleotide sequence comprising at least one of SEQ ID NOs: 45-52 or 83-90, or a complement thereof, or a variant thereof, or a truncation thereof. In some embodiments, the gRNA targets CD2 or a regulatory element thereof and comprises a polynucleotide sequence comprising at least one of SEQ ID NOs: 58-85 or 102-109, or a complement thereof, or a variant thereof, or a truncation thereof.

In some embodiments, the gRNA targets IL2RA or a regulatory element thereof. gRNAs targeting IL2RA may be used in combination with gRNAs targeting other genes. In some embodiments, the gRNA targets IL2RA or a regulatory element thereof and binds and targets a polynucleotide sequence comprising at least one of SEQ ID NOs: 92-100, or a complement thereof, or a variant thereof, or a truncation thereof. In some embodiments, the gRNA targets IL2RA or a regulatory element thereof and is encoded by a polynucleotide sequence comprising at least one of SEQ ID NOs: 92-100, or a complement thereof, or a variant thereof, or a truncation thereof. In some embodiments, the gRNA targets IL2RA or a regulatory element thereof and comprises a polynucleotide sequence comprising at least one of SEQ ID NOs: 111-119, or a complement thereof, or a variant thereof, or a truncation thereof.

In some embodiments, the gRNA targets EGFR or a regulatory element thereof. gRNAs targeting EGFR may be used in combination with gRNAs targeting other genes. In some embodiments, the gRNA targets EGFR or a regulatory element thereof and binds and targets a polynucleotide sequence comprising SEQ ID NO: 101, or a complement thereof, or a variant thereof, or a truncation thereof. In some embodiments, the gRNA targets EGFR or a regulatory element thereof and is encoded by a polynucleotide sequence comprising SEQ ID NO: 101, or a complement thereof, or a variant thereof, or a truncation thereof. In some embodiments, the gRNA targets EGFR or a regulatory element thereof and comprises a polynucleotide sequence comprising SEQ ID NO: 120, or a complement thereof, or a variant thereof, or a truncation thereof.

TABLE 1
Exemplary gRNAs targeting CD2, B2M, TIGIT,
IL2RA, or EGFR.

| | | |
|---|---|---|
| Name | gRNA sequence | gRNA |

CD2 gRNAS:

| | | |
|---|---|---|
| CD2 | AGAGGCACGTGGTTAAGCTCT | AGAGGCACGUGGUUAAGCUCU |
| gRNA1 | (SEQ ID NO: 45) | (SEQ ID NO: 58) |
| CD2 | AAAGGAACTGAAGTGAGACTG | AAAGGAACUGAAGUGAGACUG |
| gRNA2 | (SEQ ID NO: 46) | (SEQ ID NO: 59) |
| CD2 | GACTGGTGGAGTCCACACCCC | GACUGGUGGAGUCCACACCCC |
| gRNA3 | (SEQ ID NO: 47) | (SEQ ID NO: 60) |
| CD2 | TGGTTTCCTGTATAGCCCCCC | UGGUUUCCUGUAUAGCCCCCC |
| gRNA4 | (SEQ ID NO: 48) | (SEQ ID NO: 61) |
| CD2 | CTTCATGCAAAAGGAACTGAA | CUUCAUGCAAAAGGAACUGAA |
| gRNA5 | (SEQ ID NO: 49) | (SEQ ID NO: 62) |
| CD2 | GAGACTGGTGGAGTCCACACC | GAGACUGGUGGAGUCCACACC |
| gRNA6 | (SEQ ID NO: 50) | (SEQ ID NO: 63) |
| CD2 | GTTCCTTTTGCATGAAGAGCT | GUUCCUUUUGCAUGAAGAGCU |
| gRNA7 | (SEQ ID NO: 51) | (SEQ ID NO: 64) |
| CD2 | AAGGAACTGAAGTGAGACTGG | AAGGAACUGAAGUGAGACUGG |
| gRNA8 | (SEQ ID NO: 52) | (SEQ ID NO: 65) |
| CD2 | AGTGGATAAAAGGCTGTGTGG | AGUGGAUAAAAGGCUGUGUGG |
| gRNA9 | (SEQ ID NO: 83) | (SEQ ID NO: 102) |
| CD2 | CAGTGAAAGAGAAAAGGAACA | CAGUGAAAGAGAAAAGGAACA |
| gRNA10 | (SEQ ID NO: 84) | (SEQ ID NO: 103) |
| CD2 | GTTTOTTCCAAAGGTAAGCAT | GUUUCUUCCAAAGGUAAGCAU |
| gRNA11 | (SEQ ID NO: 85) | (SEQ ID NO: 104) |
| CD2 | GAGAGTCACTTTCAGGGAAAG | GAGAGUCACUUUCAGGGAAAG |
| gRNA12 | (SEQ ID NO: 86) | (SEQ ID NO: 105) |
| CD2 | AGAGAGGCTAAGTAGATCACT | AGAGAGGCUAAGUAGAUCACU |
| gRNA13 | (SEQ ID NO: 87) | (SEQ ID NO: 106) |
| CD2 | GACTGCACCTATAAAAAGCAA | GACUGCACCUAUAAAAAGCAA |
| gRNA14 | (SEQ ID NO: 88) | (SEQ ID NO: 107) |
| CD2 | TTTGGCAAAGGAGCACATCAG | UUUGGCAAAGGAGCACAUCAG |
| gRNA15 | (SEQ ID NO: 89) | (SEQ ID NO: 108) |
| CD2 | TAAATGTTCACAAGCCAATAG | UAAAUGUUCACAAGCCAAUAG |
| gRNA16 | (SEQ ID NO: 90) | (SEQ ID NO: 109) |

B2M gRNAs:

| | | |
|---|---|---|
| B2M | CACGGAGCGAGACATCTCGGC | CACGGAGCGAGACAUCUCGGC |
| gRNA1 | (SEQ ID NO: 53) | (SEQ ID NO: 66) |
| B2M | ACCTTTGGCCTACGGCGACGG | ACCUUUGGCCUACGGCGACGG |
| gRNA2 | (SEQ ID NO: 54) | (SEQ ID NO: 67) |
| B2M | GAGCACAGCTAAGGCCACGGA | GAGCACAGCUAAGGCCACGGA |
| gRNA3 | (SEQ ID NO: 55) | (SEQ ID NO: 68) |
| B2M | AGAAGGCATGCACTAGACTGG | AGAAGGCAUGCACUAGACUGG |
| gRNA4 | (SEQ ID NO: 56) | (SEQ ID NO: 69) |
| B2M | GGAGAGGAAGGACCAGAGCGG | GGAGAGGAAGGACCAGAGCGG |
| gRNA5 | (SEQ ID NO: 57) | (SEQ ID NO: 70) |

TIGIT gRNAs:

| | | |
|---|---|---|
| TIGIT | GGACAATCTCTGAGAATGAGG | GGACAAUCUCUGAGAAUGAGG |
| gRNA1 | (SEQ ID NO: 91) | (SEQ ID NO: 110) |

IL2RA gRNAs:

| | | |
|---|---|---|
| IL2RA_g1 | ATAGAGACTGGATGGACCCAC | AUAGAGACUGGAUGGACCCAC |
| | (SEQ ID NO: 92) | (SEQ ID NO: 111) |
| IL2RA_g2 | GTGGGTCCATCCAGTCTCTAT | GUGGGUCCAUCCAGUCUCUAU |
| | (SEQ ID NO: 93) | (SEQ ID NO: 112) |
| IL2RA_g3 | TAGATGGTTCCAAGAAGGGAG | UAGAUGGUUCCAAGAAGGGAG |
| | (SEQ ID NO: 94) | (SEQ ID NO: 113) |
| IL2RA_94 | TCTCACCCAGCACTTCATAAG | UCUCACCCAGCACUUCAUAAG |
| | (SEQ ID NO: 95) | (SEQ ID NO: 114) |
| IL2RA_g5 | AGATTCCCCTGCOGTTGAAGG | AGAUUCCCCUGCCGUUGAAGG |
| | (SEQ ID NO: 96) | (SEQ ID NO: 115) |
| IL2RA_g6 | TCAATTGCTGGAGGTGTGGGC | UCAAUUGCUGGAGGUGUGGGC |
| | (SEQ ID NO: 97) | (SEQ ID NO: 116) |
| IL2RA_g7 | ACTCAGCTTATGAAGTGCTGG | ACUCAGCUUAUGAAGUGCUGG |
| | (SEQ ID NO: 98) | (SEQ ID NO: 117) |
| IL2RA_g8 | GTGCTGGGTGAGACCACTGCC | GUGCUGGGUGAGACCACUGCC |
| | (SEQ ID NO: 99) | (SEQ ID NO: 118) |
| IL2RA_g9 | TTTTATGGGOGTAGCTGAAGA | UUUUAUGGGCGUAGCUGAAGA |
| | (SEQ ID NO: 100) | (SEQ ID NO: 119) |

EGFR gRNAs:

| | | |
|---|---|---|
| EGFR | TCGGGAGGAGCAGAGGAGGAG | UCGGGAGGAGCAGAGGAGGAG |
| gRNA1 | (SEQ ID NO: 101) | (SEQ ID NO: 120) |

In some embodiments, the gRNA targets a gene in an immune cell. The gene may be expression ubiquitously. The gene may be expressed specifically in an immune cell. In some embodiments, the immune cell is a T cell. In some embodiments, the immune cell is a primary immune cell. The gRNA may target a sequence within 100, 200, 300, 400, 500, 600, 700, 800, or 900 base pairs of the transcriptional start site of the gene. In some embodiments, the gRNA targets a sequence within 500 base pairs of the transcriptional start site of the gene.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

d. Repair Pathways

The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA when the Cas9 protein or Cas9 fusion protein has nuclease activity. This DNA cleavage may stimulate the natural DNA-repair machinery, leading to one of two possible repair pathways: homology-directed repair (HDR) or the non-homologous end joining (NHEJ) pathway.

i) Homology-Directed Repair (HDR)

Restoration of protein expression from a gene may involve homology-directed repair (HDR). A donor template may be administered to a cell. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

ii) NHEJ

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 4. REPORTER PROTEIN

In some embodiments, the DNA targeting compositions or CRISPR/Cas9 systems include at least one reporter protein. A polynucleotide sequence encoding the reporter protein may be operably linked to the polynucleotide sequence encoding the Cas9 protein or Cas9 fusion protein. The reporter protein may include any protein or peptide that is suitably detectable, such as, by fluorescence, chemiluminescence, enzyme activity such as beta galactosidase or alkaline phosphatase, and/or antibody binding detection. The reporter protein may comprise a fluorescent protein. The reporter protein may comprise a protein or peptide detectable with an antibody. For example, the reporter protein may comprise GFP, YFP, RFP, CFP, DsRed, luciferase, and/or Thy1.

In some embodiments, the systems detailed herein include a polynucleotide sequence encoding a 2A self-cleaving peptide operably linked to the polynucleotide sequence encoding the Cas9 protein or Cas9 fusion protein and to the polynucleotide sequence encoding the reporter protein. The T2A polynucleotide sequence may be between the polynucleotide sequence encoding the Cas9 protein or Cas9 fusion protein and the polynucleotide sequence encoding the reporter protein.

### 5. GENETIC CONSTRUCTS

The CRISPR/Cas9-based gene editing system may be encoded by or comprised within a genetic construct. In some embodiments, provided herein is an isolated polynucleotide encoding a CRISPR/Cas9 system as detailed herein. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes at least one gRNA molecule, and a second genetic construct encodes a Cas9 molecule or Cas9 fusion protein. In some embodiments, the CRISPR/Cas9-based gene editing system comprises a first vector comprising a polynucleotide sequence encoding a fusion protein, and a second vector comprising a polynucleotide sequence encoding at least one gRNA. In some embodiments, the CRISPR/Cas9-based gene editing system comprises a single vector comprising a polynucleotide sequence encoding a fusion protein and a polynucleotide sequence encoding at least one gRNA.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual. Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The initiation and termination codon may be in frame with the CRISPR/Cas-based gene editing system coding sequence. The vector may also comprise a promoter that is operably linked to the CRISPR/Cas-based gene editing system coding sequence. In some embodiments, the promoter is operably linked to a polynucleotide encoding a Cas9 protein or Cas9 fusion protein. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example. In some embodiments, the promoter is a human Pol III U6 promoter upstream of and driving expression of the polynucleotide sequence encoding the gRNA. In some embodiments, the human Pol III U6 promoter and the polynucleotide sequence encoding the gRNA are orientated in the opposite direction from the polynucleotide sequence encoding the fusion protein.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is an immune cell. The immune cell may be a human immune cell. In some embodiments, the immune cell is T cell.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-846). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

The genetic construct may comprise a polynucleotide sequence of SEQ ID NO: 71 or 72.

### 6. PHARMACEUTICAL COMPOSITIONS

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 7. ADMINISTRATION

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells.

### 8. KITS

Provided herein is a kit, which may be used to modulate expression of a gene in a cell such as an immune cell. The kit comprises genetic constructs or a composition comprising the same, as described above, and instructions for using said composition. In some embodiments, the kit comprises at least one gRNA comprising a polynucleotide sequence selected from SEQ ID NOs: 58-70, a complement thereof, a variant thereof, or a fragment thereof, or encoded by or targeting a polynucleotide sequence selected from SEQ ID NOs: 45-57, and instructions for using the CRISPR/Cas-based gene editing system. In some embodiments, the kit comprises a polynucleotide sequence encoding a Cas9 protein or a Cas9 fusion protein, and instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising the same for modulating expression of a gene in a cell such as an immune cell may include a modified AAV vector that includes a gRNA molecule(s) and a Cas9 protein or fusion protein, as described above, that specifically binds a region of gene in an immune cell. The CRISPR/Cas-based gene editing system, as described above, may be included in the kit to specifically bind and target a particular region, for example, a region of the CD2 or B2M gene.

### 9. METHODS

a. Methods of Modulating Expression of a Gene in a Cell

Provided herein are methods of modulating expression of a gene in a cell. The methods may include administering to the cell a CRISPR/Cas9 system as detailed herein, an isolated polynucleotide as detailed herein, a vector as detailed herein, a cell as detailed herein, or vector as detailed herein. In some embodiments, the cell is an immune cell. In some embodiments, the immune cell is a T cell.

Further provided herein are methods of reducing B2M expression of a gene in a cell. The methods may include administering to the cell a CRISPR/Cas9 system as detailed herein, an isolated polynucleotide as detailed herein, a vector as detailed herein, a cell as detailed herein, or vector as detailed herein. The gRNA may target B2M or a regulatory element thereof. In some embodiments, the cell is an immune cell. In some embodiments, the immune cell is a T cell. The second polypeptide domain of the Cas fusion protein may have transcription repression activity.

Further provided herein are methods of reducing immunological activity of a cell. The methods may include administering to the cell a CRISPR/Cas9 system as detailed herein, an isolated polynucleotide as detailed herein, a vector as detailed herein, a cell as detailed herein, or vector as detailed herein. The gRNA may target B2M or a gene regulatory element thereof. The second polypeptide domain of the Cas fusion protein may have transcription repression activity. In some embodiments, the cell is an immune cell. In some embodiments, the immune cell is a T cell.

Further provided herein are methods of reducing TIGIT expression in a cell. The methods may include administering to the cell a CRISPR/Cas9 system as detailed herein, an isolated polynucleotide as detailed herein, a vector as detailed herein, a cell as detailed herein, or vector as detailed herein. The gRNA may target TIGIT or a gene regulatory element thereof. The second polypeptide domain of the Cas fusion protein may have transcription repression activity. In some embodiments, the cell is an immune cell. In some embodiments, the immune cell is a T cell.

Further provided herein are methods of reducing CD2 expression in a cell. The methods may include administering to the cell a CRISPR/Cas9 system as detailed herein, an isolated polynucleotide as detailed herein, a vector as detailed herein, a cell as detailed herein, or vector as detailed herein. The gRNA may target CD2 or a gene regulatory element thereof. The second polypeptide domain of the Cas fusion protein may have transcription repression activity. In some embodiments, the cell is an immune cell. In some embodiments, the immune cell is a T cell.

Further provided herein are methods of increasing CD2 expression in a cell. The methods may include administering to the cell a CRISPR/Cas9 system as detailed herein, an isolated polynucleotide as detailed herein, a vector as detailed herein, a cell as detailed herein, or vector as detailed herein. The gRNA may target CD2 or a gene regulatory element thereof. The second polypeptide domain of the Cas fusion protein may have transcription activation activity. In some embodiments, the cell is an immune cell. In some embodiments, the immune cell is a T cell.

Further provided herein are methods of increasing EGFR expression in a cell. The methods may include administering to the cell a CRISPR/Cas9 system as detailed herein, an isolated polynucleotide as detailed herein, a vector as detailed herein, a cell as detailed herein, or vector as detailed herein. The gRNA may target EGFR or a gene regulatory element thereof. The second polypeptide domain of the Cas fusion protein may have transcription activation activity. In some embodiments, the cell is an immune cell. In some embodiments, the immune cell is a T cell.

Further provided herein are methods of increasing IL2RA expression in a cell. The methods may include administering to the cell a CRISPR/Cas9 system as detailed herein, an isolated polynucleotide as detailed herein, a vector as detailed herein, a cell as detailed herein, or vector as detailed herein. The gRNA may target IL2RA or a gene regulatory element thereof. The second polypeptide domain of the Cas fusion protein may have transcription activation activity. In some embodiments, the cell is an immune cell. In some embodiments, the immune cell is a T cell.

b. Methods of Treating a Subject Having a Disease

Provided herein are methods of treating a subject having a disease. The methods may include administering to the subject a CRISPR/Cas9 system as detailed herein, an isolated polynucleotide as detailed herein, a vector as detailed herein, a cell as detailed herein, or vector as detailed herein. The disease may comprise cancer, an autoimmune disease, or a viral infection.

Further provided herein are methods of increasing an immune cell's ability to kill a cancer cell. The methods may include administering to the cell a CRISPR/Cas9 system as detailed herein, an isolated polynucleotide as detailed herein, a vector as detailed herein, a cell as detailed herein, or vector as detailed herein. The gRNA may target TIGIT or a gene regulatory element thereof. The second polypeptide domain of the Cas fusion protein may have transcription repression activity. In some embodiments, the cell is an immune cell. In some embodiments, the immune cell is a T cell.

c. Methods of Screening for Gene Regulatory Elements

Provided herein are methods of screening for one or more putative gene regulatory elements in a genome that modulate a phenotype of an immune cell. The methods may include contacting a plurality of modified target immune cells with a library of gRNAs, each gRNA targeting a gene regulatory element in an immune cell, thereby generating a plurality of test immune cells; selecting a population of test immune cells or an organism having a modulated phenotype; quantitating the frequency of the gRNAs within the population of selected immune cells or the organism, wherein the gRNAs that target gene regulatory elements that modulate the phenotype are overrepresented or underrepresented in the selected immune cells; and identifying and characterizing the gRNAs within the population of selected immune cells or the organism thereby identifying the gene regulatory elements that modulate the phenotype. In some embodiments, the modified target immune cell or organism comprises a fusion protein, the fusion protein comprising a first polypeptide domain comprising a nuclease-inactivated Staphylococcus aureus Cas9 protein (dSaCas9) and a second polypeptide domain having an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity. In some embodiments, the immune cell is a T cell.

Provided herein is a method of screening a library of gRNAs for modulation of gene expression in a cell. The method may include generating a library of vectors with a library of gRNAs, each gRNA targeting a target gene in a cell, the library of vectors comprising: a polynucleotide sequence encoding a fusion protein, wherein the fusion protein comprises two heterologous polypeptide domains, wherein the first polypeptide domain comprises a nuclease-inactivated Staphylococcus aureus Cas9 protein (dSaCas9), and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; a polynucleotide sequence encoding a reporter protein operably linked to the polynucleotide sequence encoding the fusion protein; and a polynucleotide sequence encoding one of the gRNAs. The method may further include transducing a plurality of cells with the library of vectors; culturing the transduced cells; sorting the cultured cells based on the growth of the cells or on the level of expression of the reporter protein; and sequencing the gRNA from each sorted cell. In some embodiments, the reporter protein comprises a fluorescent protein and/or a protein detectable with an antibody, and wherein the cultured cells are sorted based on the level of expression of the reporter protein. In some embodiments, the cell is an immune cell. In some embodiments, the immune cell is a T cell. In some embodiments, the library of vectors further comprises a polynucleotide sequence encoding a 2A self-cleaving peptide operably linked to the polynucleotide sequence encoding the fusion protein and to the polynucleotide sequence encoding the reporter protein, wherein the polynucleotide sequence encoding a 2A self-cleaving peptide is between the polynucleotide sequence encoding the fusion protein and the polynucleotide sequence encoding the reporter protein. The method may further include identifying the target gene of the gRNA that was sequenced. The method may further include modulating the level of the gene target discovered or modulating the activity of the protein produced from the gene target discovered for enhancing properties of a cell therapy.

In other embodiments, the method of screening a library of gRNAs for modulation of gene expression in a cell may include generating a library of vectors with a library of gRNAs, each gRNA targeting a target gene or a regulatory element thereof in a cell, the library of vectors comprising a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity, and a polynucleotide sequence encoding one of the gRNAs; transducing a plurality of cells with the library of gRNAs; culturing the transduced cells; capturing the gRNA from the transduced cells; and sequencing the gRNA from each transduced cell captured. In some embodiments, the gRNA from the transduced cells is captured with single cell technology. Single cell technology may include, for example, systems and kits from 10× Genomics (Pleasanton, CA). For example, the single cell technology may include systems and kits for Chromium Single Cell Gene Expression from 10× Genomics (Pleasanton, CA). In some embodiments, the method further comprises determining the level of mRNA expression and/or the level of protein expression in the transduced cells. In some embodiments, the method further includes grouping transduced cells having the same gRNA, and comparing the target gene expression of transduced cells having the same gRNA, at the mRNA and/or protein level, to the target gene expression of cells without the same gRNA. In some embodiments, the method further includes identifying the target gene of the gRNA sequenced. In some embodiments, the method further includes modulating the level of the gene target or modulating the activity of the protein produced from the gene target for enhancing properties of a cell therapy. In some embodiments, the cell is an immune cell. In some embodiments, the immune cell is a T cell. In some embodiments the first polypeptide domain comprises a Staphylococcus aureus Cas9 protein (SaCas9). In some embodiments, the first polypeptide domain comprises a nuclease-inactivated Staphylococcus aureus Cas9 protein (dSaCas9).

### 10. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### CRISPR Interference (CRISPRi) Lentiviral System

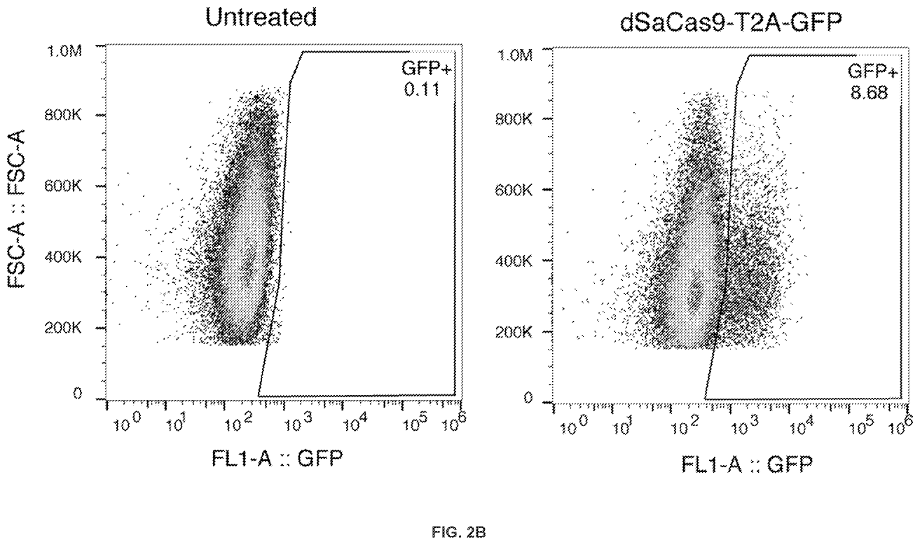

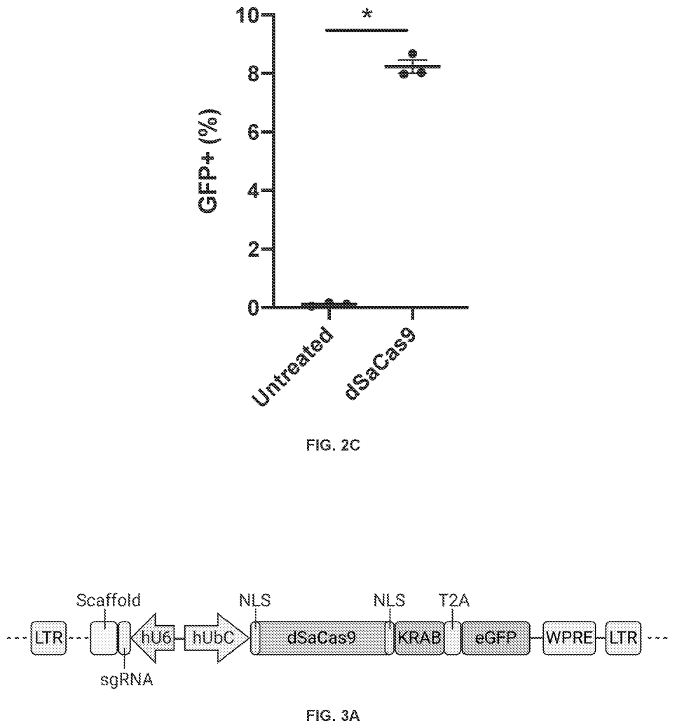

A CRISPRi construct was developed as shown in FIG. 2A. The all-in-one CRISPR interference (CRISPRi) lentiviral system features a nuclease-dead Staphylococcus aureus Cas9 (dSaCas9) tethered to a repressive protein domain (KRAB). The human ubiquitin promoter drives expression of the dSaCas9 fused to a KRAB repressive domain, which is linked to GFP expression by a 2A self-cleaving peptide sequence.

T cells were transduced with the CRISPRi vector and assayed by flow cytometry for expression of GFP on day 4 after transduction. Results of treated and untreated T cells are shown in FIG. 2B. Shown in FIG. 2C are summary statistics of the GFP+ cells (%) for untreated and transduced cells. A two-tailed t-test was conducted to determine statistical significance. An asterisk denotes P<0.05. Expression of GFP was present only in treated cells, indicating expression of the dSaCas9 fused to a KRAB repressive domain in the treated cells.

### Example 2

### CRISPRi CD2 Screen

Proof-of-concept robust gene repression of the highly expressed surface protein cluster of differentiation 2 (CD2) was demonstrated using a gRNA library and a CRISPRi construct. CD2 is a surface marker that is highly and ubiquitously expressed. A SaCas9 CRISPRi gRNA library against CD2 was generated. To screen hundreds to thousands of single gRNAs in a pooled format, gRNA libraries targeting a 1.05 kb region centered around the transcriptional start site (TSS) of CD2 (saturation libraries spanning −400 to +650 of the TSS). The library included 141 targeting gRNAs. The library also contained 250 non-targeting gRNAs as negative controls.

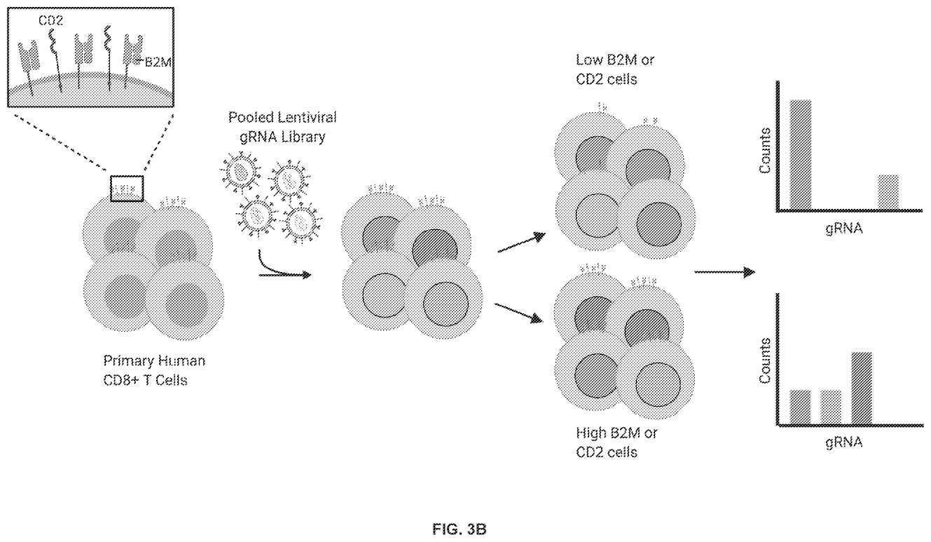

A schematic of the CRISPRi construct (Lentiviral All-in-One SaCas9 Vector) is detailed in FIG. 3A. The CRISPRi construct included a human ubiquitin promoter to drive expression of dSaCas9 fused to a KRAB repressive domain, which was linked to GFP expression by a 2A self-cleaving peptide sequence to enable sorting of transduced cells. A human Pol III U6 promoter orientated in the opposite direction drove expression of the single gRNA. The gRNA recruited dSaCas9 to the target genomic DNA site. The library was cloned into the gRNA expression cassette of the single lentiviral vector encoding dSaCas9 fused to a KRAB repressor domain (dSaCas9KRAB) and linked to GFP by a 2A sequence.

A schematic of the screening pipeline is shown in FIG. 3B. Primary Human CD8+ T cells were first isolated and activated from peripheral blood mononuclear cells (PBMCS). Next, activated primary human T cells were transduced with the pooled gRNA library targeting a 1.05 kb window around the TSS of CD2 and cultured for 9 days. T cells were then stained with a CD2 antibody and sorted into low and high (10%) bins of CD2 expression (bottom and top 10% bins) based on antibody signal with fluorescence-activated cell sorting (FACS). The genomic DNA was isolated and purified from the sorted populations, the gRNA cassette was amplified from the genomic DNA, the gRNA libraries were sequenced, and DeSeq2 was used to identify differentially abundant gRNAs in either bin.

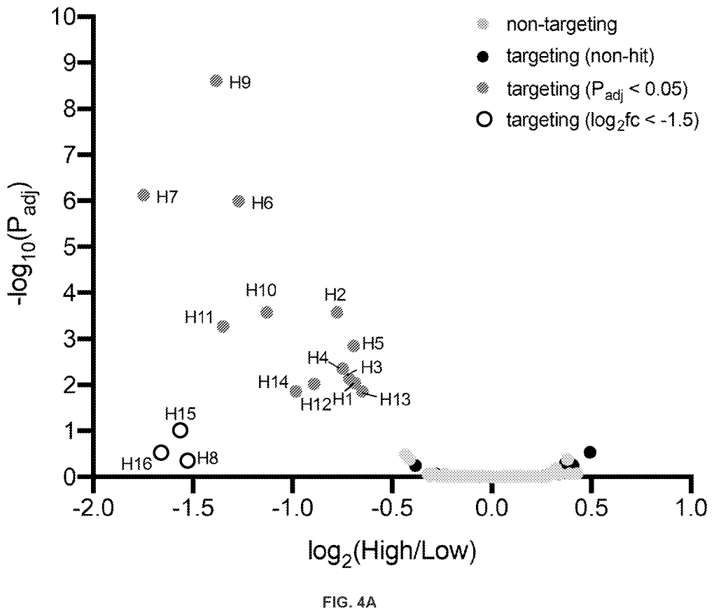

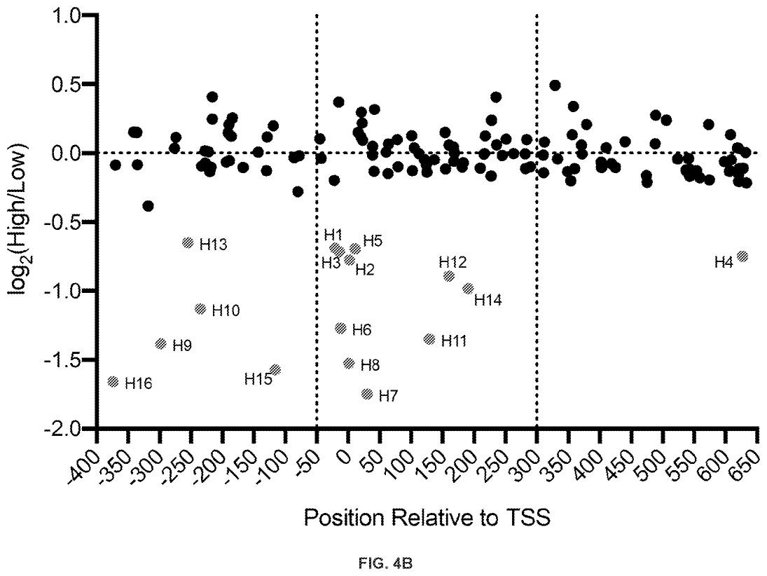

Robust gene repression of CD2 was demonstrated. Only targeting gRNAs—the majority of which fell within an optimal window for CRISPRi applications—emerged as hits. A volcano plot (−log 10 of adjusted P values vs fold-change in counts between high and low bins) of gRNAs is shown in FIG. 4A. Non-targeting gRNAs are labeled in light gray, and targeting gRNAs are in dark gray (with statistically significant gRNAs in open circles). The major of gRNA hits fell within a defined optimal window for repression. Shown in FIG. 4B is the fold change vs. gRNA positioning relative to TSS. gRNAs that fell within the window and were enriched (but not statistically significant) were also included for validation. Dashed boxes indicate which gRNAs were then individually cloned and validated. 16 CD2-targeting gRNAs were found enriched in the low bin (see TABLE 1 above). Importantly, there was no enrichment of non-targeting gRNAs in either bin, indicating that the screens had minimal noise and that the observed effects were gRNA-dependent.

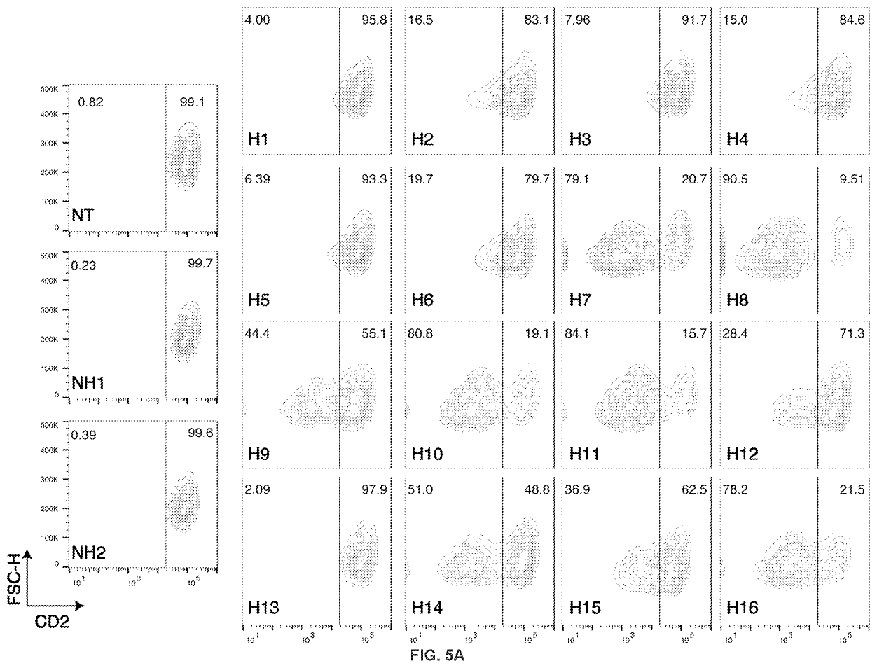

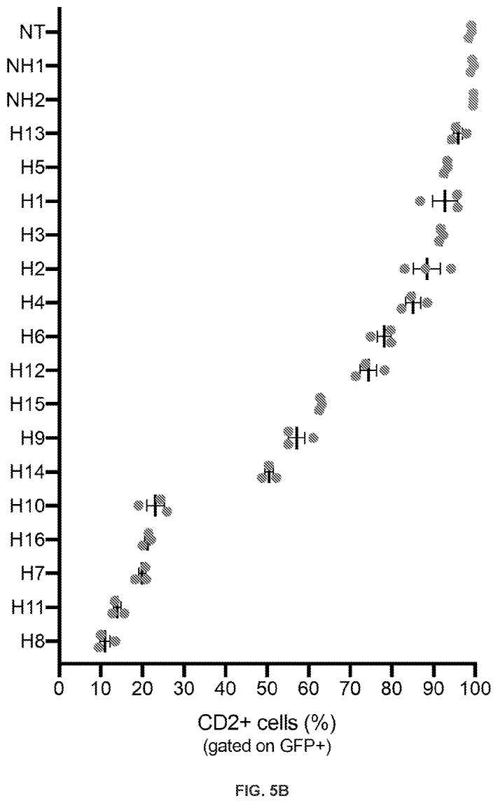

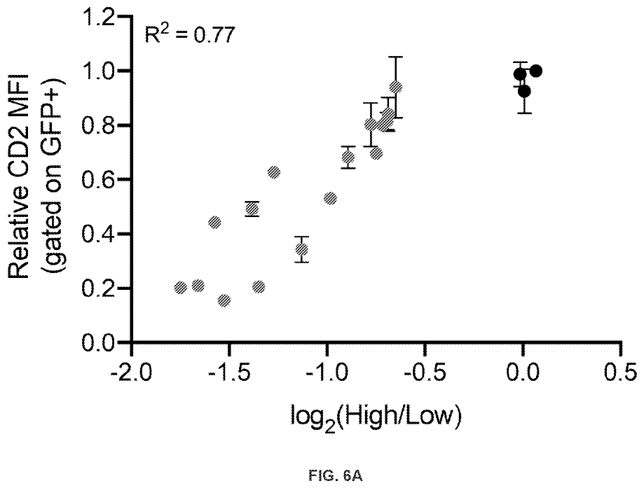

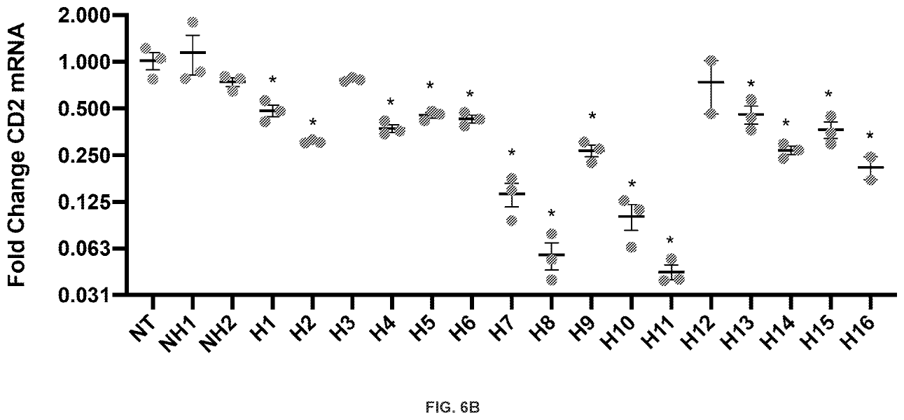

To functionally validate these hits, eight individual CD2-targeting gRNAs were cloned into the same lentiviral vector and delivered them to primary T cells. This lentiviral backbone also contained a fluorophore to enable differentiation of non-transduced and transduced cells. Using flow cytometry, a marked shift in CD2 expression was noted for all eight gRNAs in only transduced cells relative to the nontargeting gRNA. Representative density plots of CD2 repression for each gRNA are shown in FIG. 5A. FIG. 5B is a graph showing the percentage of CD2+ cells for each gRNA. The CD2-low gate was set with non-targeting control gRNA. Shown in FIG. 6A is a graph showing gRNA activity was correlated with log 2(fc) from the screen. The graph compares CD2 protein levels (MRI=mean fluorescence intensity) on the y-axis for each gRNA to the strength of depletion in the screen for that gRNA. On the x-axis, log 2(fc) is log 2(fold-change), wherein “fold-change” is the difference in gRNA abundance in the screen between the CD2 high and CD2 low populations. The transduced cells (GFP+) were sorted, RNA was isolated and reverse transcribed into cDNA, and RT-qPCR for CD2 was performed to assay gene expression at the transcription level. Shown in FIG. 6B are results from RT-qPCR of CD2 within transduced cells, showing the fold change in CD2 mRNA for each gRNA. The ddct normalization method was used with GAPDH being the householding gene and all CD2 expression levels relative to the non-targeting control. One-way ANOVA was conducted to determine statistical significance for FIG. 6A and FIG. 6B. Combining these metrics, the mRNA levels were highly correlated to the protein levels and the best performing gRNAs achieved 70-80% repression.

### Example 3

### CRISPRi B2M Screen

Proof-of-concept robust regulation of gene repression of the highly expressed surface protein beta-2 microglobulin (B2M) was demonstrated using a gRNA library and a CRISPRi construct. B2M is a surface marker that is highly and ubiquitously expressed. A SaCas9 CRISPRi gRNA library against the highly expressed surface protein B2M was generated, targeting a 1.05 kb region centered around the transcriptional start site (TSS) of B2M (saturation libraries spanning −400 to +650 of the TSS). The library included 217 targeting gRNAs. The library also contained 250 non-targeting gRNAs as negative controls.

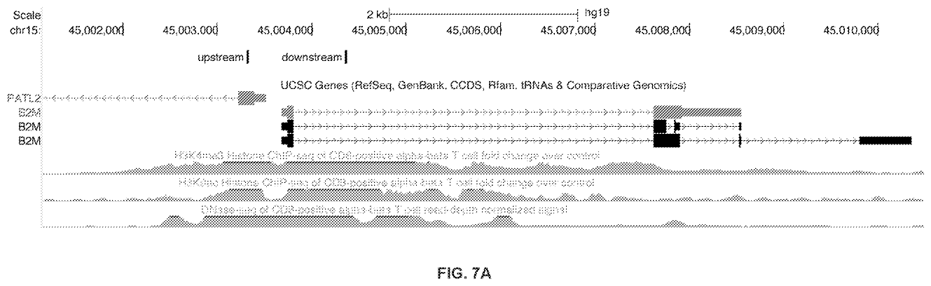

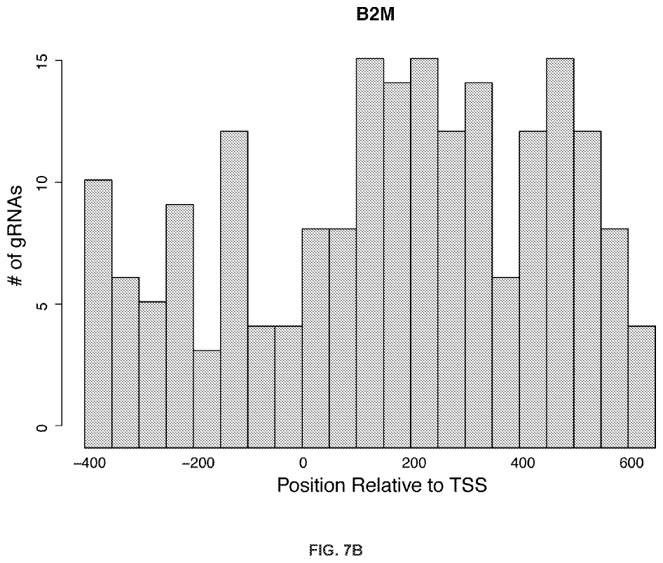

A schematic diagram for the design of the B2M gRNAs is shown in FIG. 7A, with a UCSC genome browser track with upstream and downstream of most gRNAs targeting B2M annotated. DNase-seq and ChIP-seq tracks of histone marks associated with active transcription and open chromatin are also displayed. Shown in FIG. 7B is a histogram of gRNA abundance relative to the TSS (B2M TPM>20.000).

A schematic of the CRISPRi construct (Lentiviral All-in-One SaCas9 Vector) is detailed in FIG. 3A. The CRISPRi construct included a human ubiquitin promoter to drive expression of dSaCas9 fused to a KRAB repressive domain, which was linked to GFP expression by a 2A self-cleaving peptide sequence to enable sorting of transduced cells. A human Pol III U6 promoter orientated in the opposite direction drove expression of the single gRNA. The gRNA recruited dSaCas9 to the target genomic DNA site. The library was cloned into the gRNA expression cassette of the single lentiviral vector encoding dSaCas9 fused to a KRAB repressor domain (dSaCas9KRAB) and linked to GFP by a 2A sequence.

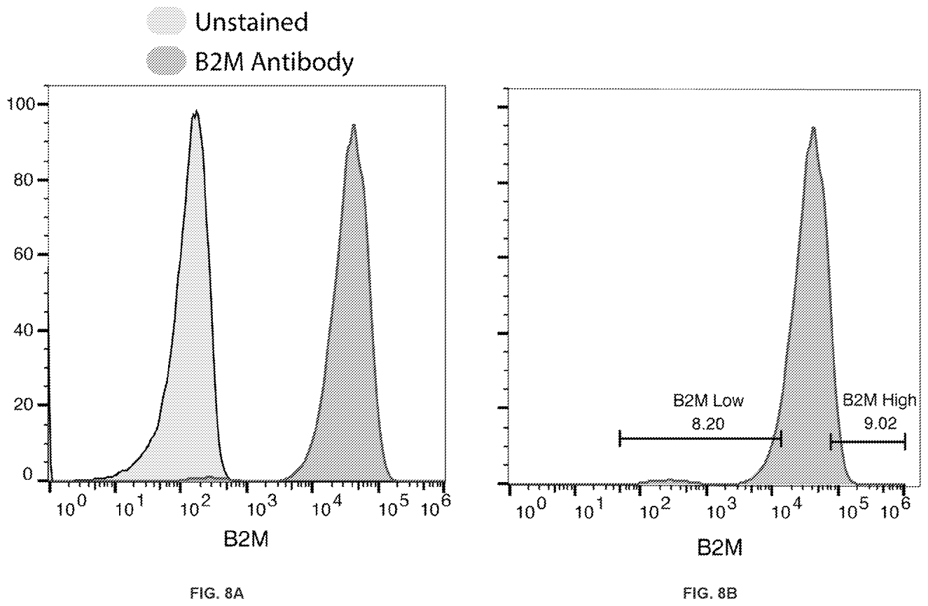

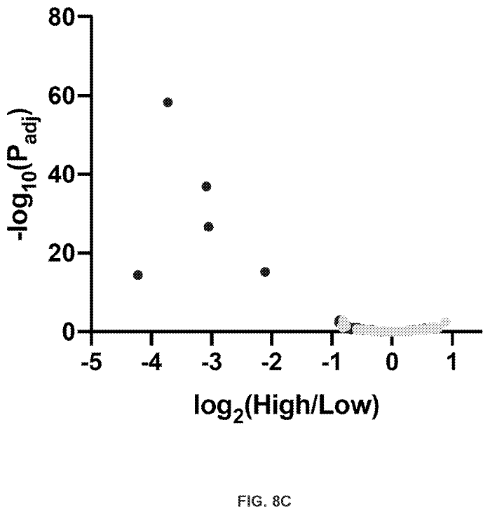

A schematic of the screening pipeline is shown in FIG. 3B. Primary Human CD8+ T cells were first isolated and activated from peripheral blood mononuclear cells (PBMCS). Next, activated primary human T cells were transduced with the pooled gRNA library targeting a 1.05 kb window around the TSS of B2M and cultured for 9 days. T cells were then stained with a B2M antibody and sorted into low and high (10%) bins of B2M expression (bottom and top 10% bins) based on antibody signal with fluorescence-activated cell sorting (FACS). Shown in FIG. 8A is the B2M distribution for unstained and stained T cells. Shown in FIG. 8B are the lower and upper ˜10% tails of B2M-expressing cells that were sorted off into low and high bins. The genomic DNA was then isolated and purified from the sorted populations, the gRNA cassette was amplified from genomic DNA, the gRNA libraries were deep sequenced, and DeSeq2 was used to identify differentially abundant gRNAs in either bin.

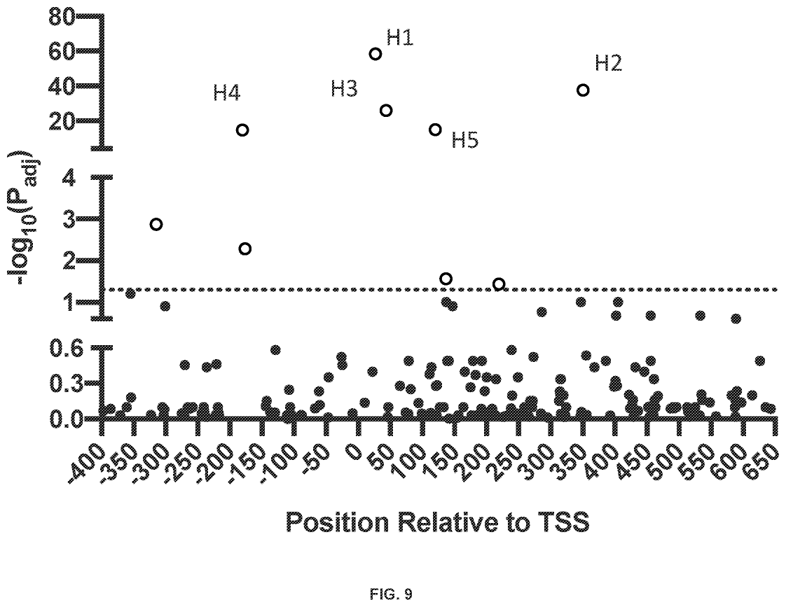

Robust gene repression of B2M was demonstrated overtime. Only targeting gRNAs—the majority of which fell within a defined optimal window for CRISPRi applications—emerged as hits. A volcano plot (−log 10 of adjusted P values vs fold-change in counts between high and low bins) of gRNAs is shown in FIG. 8C. Non-targeting gRNAs are labeled in light gray, targeting gRNAs are in dark gray. FIG. 9 is a plot showing statistical significance vs. gRNA positioning relative to TSS. Open circles denote statistically significant gRNAs (P_{adjusted}<0.05). The top 5 gRNA hits (labeled H1-H5) were cloned for individual validation. 5 B2M-targeting gRNAs were found enriched in the low bin (see TABLE 1 above). Importantly, there was no enrichment of non-targeting gRNAs in either bin, indicating that the screens had minimal noise and that the observed effects were gRNA-dependent.

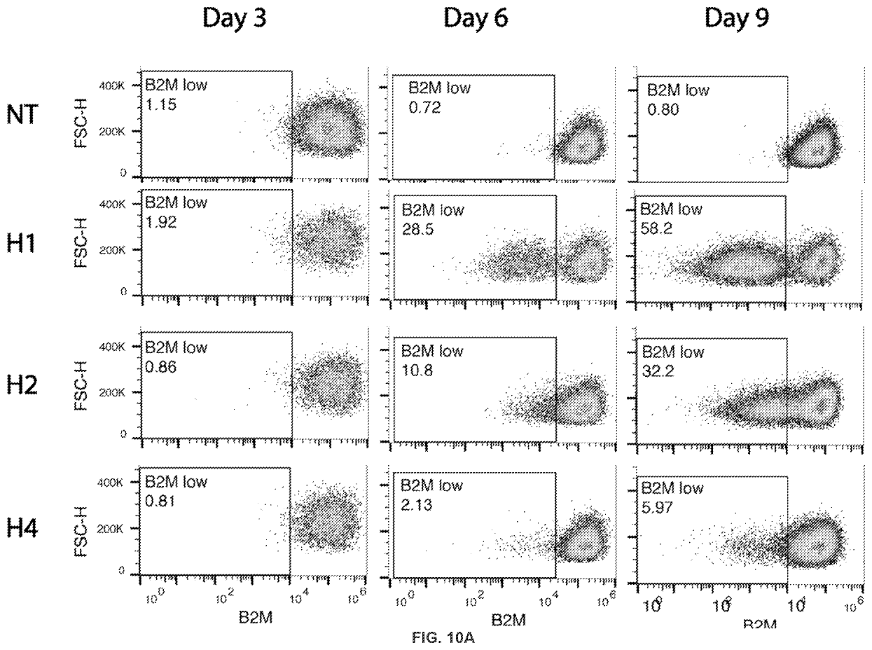

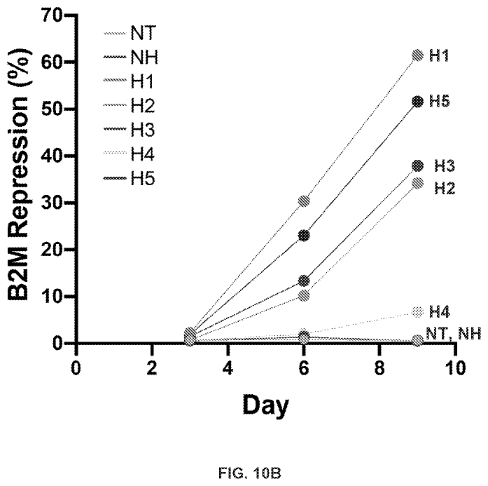

To functionally validate these hits, individual B2M-targeting gRNAs were cloned into the same lentiviral vector and delivered them to primary T cells. This lentiviral backbone also contained a fluorophore to enable differentiation of non-transduced and transduced cells. Using flow cytometry, a shift was noted in B2M expression for all gRNAs in only transduced cells relative to the nontargeting gRNA. Shown in FIG. 10A are representative density plots of B2M repression for non-targeting (NT), H1, H2, and H4 across 3 time points (day 3, 6, and 9). The B2M low gate was set with the non-targeting control. gRNAs differed markedly in their kinetics of repression. Shown in FIG. 10B is a scatter plot of B2M repression overtime for each gRNA. Each solid point/line represents the averaged percentage of silenced B2M cells across 3 replicates (with individual replicates being plotted opaque). Shown in FIG. 11A are summary statistics of the percentage of cells repressing B2M across replicates for each gRNA. The transduced cells (Thy1.1+) were then sorted, RNA was isolated and reverse transcribed into cDNA, and RT-qPCR for B2M was performed to assay gene expression at the transcription level. Shown in FIG. 11B are results from RT-qPCR of B2M within transduced cells. The ddct normalization method was used with GAPDH being the householding gene and all B2M expression levels relative to the non-targeting control. One-way ANOVA was used to determine statistical significance for FIG. 11A and FIG. 11B.

Overall, this CRISPRi platform could be readily extended to other known therapeutic gene targets to improve both the effector response and persistence of cell-based immunotherapies.

### Example 4

### Single Cell CRISPRi CD2 Screen

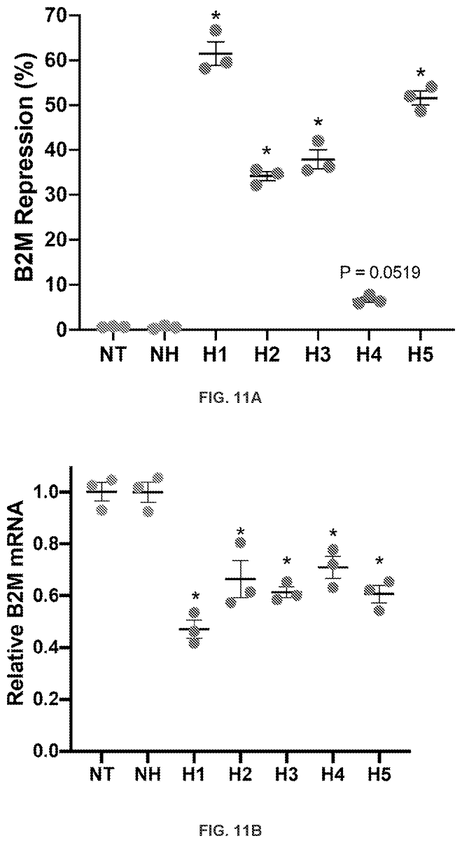

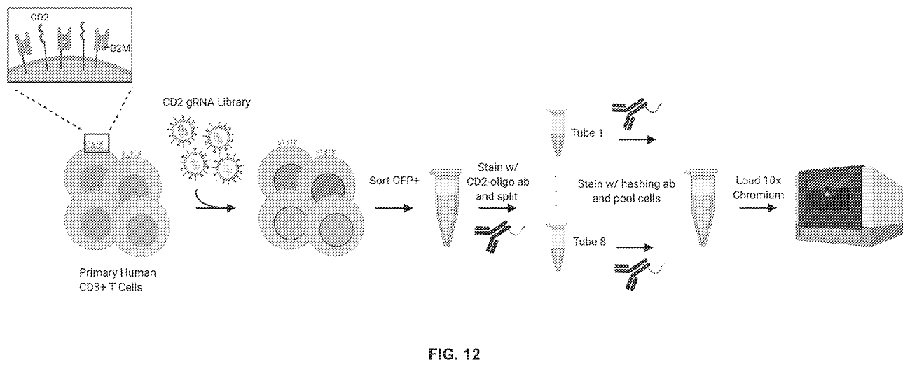

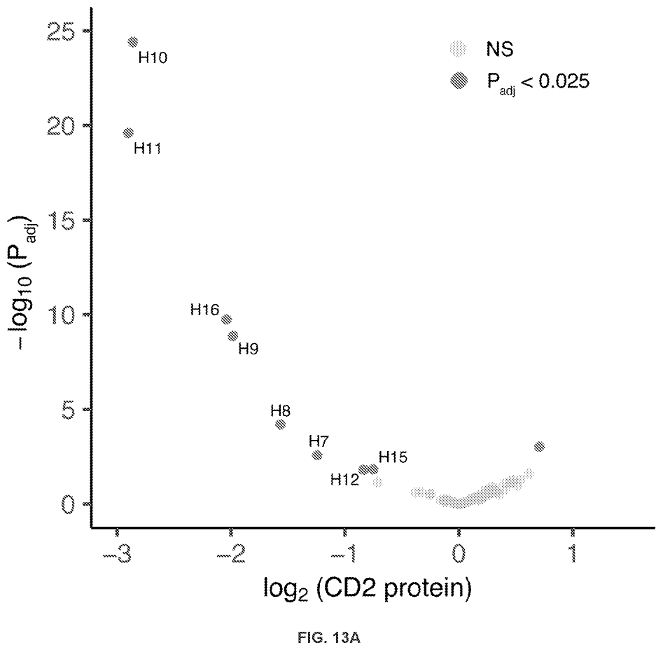

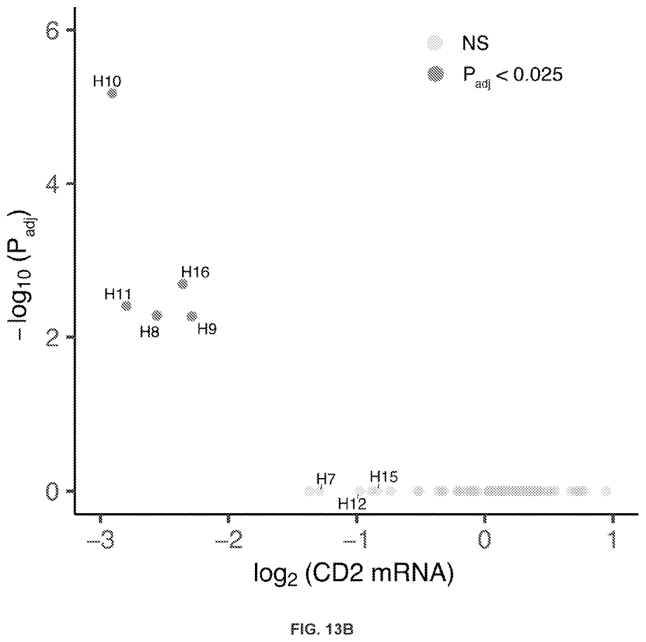

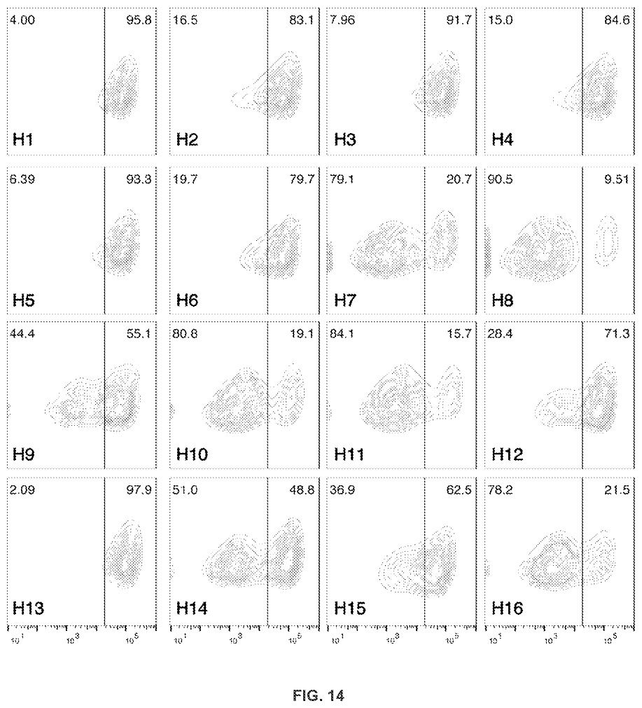

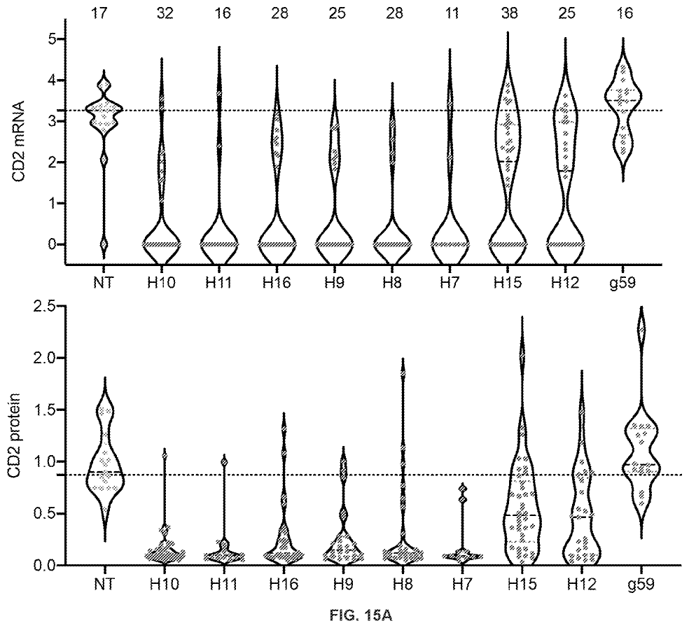

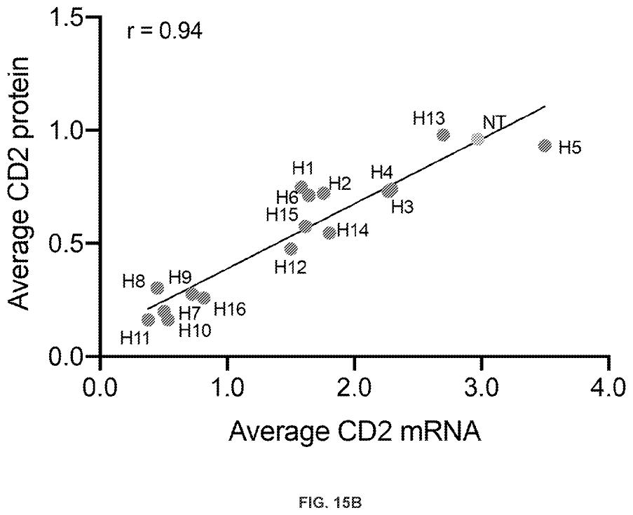

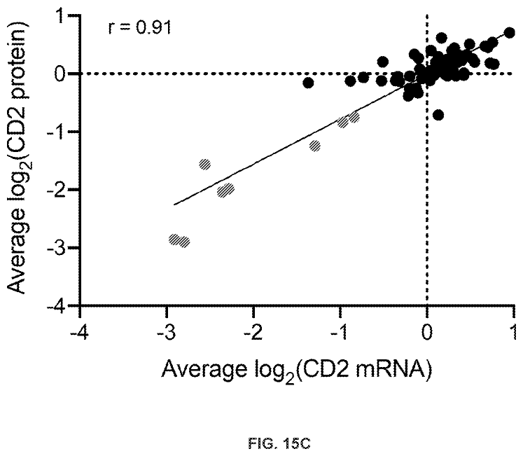

10× Genomics 5′ single-cell sequencing platform (Pleasanton, CA) was adapted to capture SaCas9 gRNAs and capture their effects on gene expression at the RNA and protein level (FIG. 12). Our previously validated CD2 gRNA library (see Example 2) was used for the pilot screen. To capture non-polyadenylated gRNA transcripts, a custom reverse transcription primer with an annealing sequence complementary to the scaffold region of SaCas9 gRNAs and a PCR handle for subsequent amplification was spiked in. This enabled the assignment of a particular gRNA or combination of gRNAs to each cell. The oligonucleotides used for the single cell screen are shown in TABLE 2. In addition to recovering gRNAs and mRNA transcripts from single cells, barcoding technology was used to quantify CD2 protein levels by staining the cells with a DNA barcoded CD2 antibody compatible with 10× Genomics's cell barcoded beads. Using this multimodal information (gRNA, mRNA, and protein) from each profiled cell, cells were aggregated based on gRNA identity, and CD2 mRNA and protein levels of cells with a targeting gRNA were compared to all cells with only non-targeting gRNAs (FIG. 13A-FIG. 13B). Differential analysis of CD2 at the mRNA and protein level revealed previously validated potent CD2 gRNA hits (gRNA7, gRNA8, gRNA9, gRNA10, gRNA11, gRNA12, gRNA15, gRNA16; see FIG. 14, FIG. 15A, FIG. 15B, FIG. 15C), demonstrating the feasibility of capturing and detecting SaCas9 gRNA effects with this approach.

TABLE 2
Oligonucleotides used in the CD2 single cell screen.

| Oligo | | |
|---|---|---|
| Name | Oligo Sequence | Oligo Function |
| Custom SA | AGCAAGTGAGAAGCATCGTGTCaaaatct | Custom reverse |
| gRNA RT | cgCcaacaagttg | transcription primer for |
| primer | (SEQ ID NO: 121) | SaCas9 scaffold |
| | | containing a PCR |
| | | handle for amplification |
| | | after GEMS are broken |
| | | (enables direct bead |
| | | capture of guides) |
| gRNA tag | AGCAAGTGAGAAGCATCGTG*T*C | Reverse primer for |
| additive | (SEQ ID NO: 122) | guide-specific |
| primer | | amplification during |
| | | cDNA amplification, |
| | | complementary to PCR |
| | | handle added during RT |
| gRNA sclib | AATGATACGGCGACCACCGAGATCTACAC | Forward primer for |
| construction | TCTTTCCCTACACGACGC*T*C | scRNA-seq Sa guide |
| FW | (SEQ ID NO: 123) | RNA library |
| | | construction, contains |
| | | P5 and part of the |
| | | TruSeq Read 1 |
| | | sequence |
| gRNA sclib | CAAGCAGAAGACGGCATACGAGATNNNNN | Reverse primer for |
| construction | NNNNNGTCTCGTGGGCTCGGAGATGTGTA | scRNA-seq Sa guide |
| REV | TAAGAGACAGtgtttccagagtactaaa* | RNA library |
| | a*c (SEQ ID NO: 124) | construction, contains |
| | | P7, 10 bp 17 index, |
| | | Nextera Read 2N, and |
| | | sequence |
| | | complementary to |
| | | SaCas9 scaffold |

*denotes a phosphorothioate bond; N denotes any of the 4 nucleotides (A, C, T, G).

### Example 5

### dSaCas9-Epigenome Effectors Function in Tumor-Infiltrating Lymphocytes (TILs)

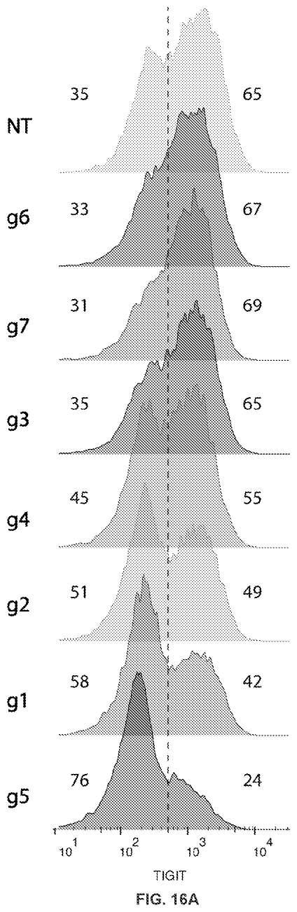

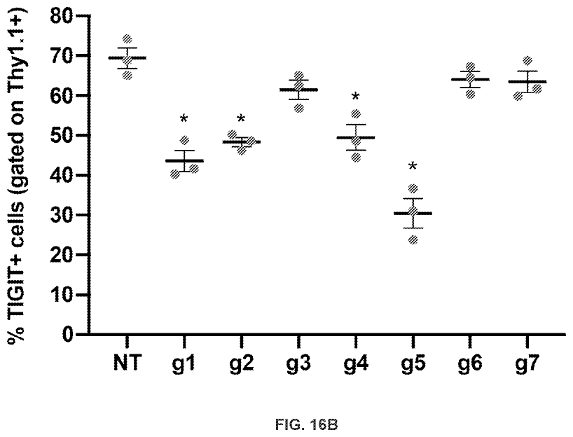

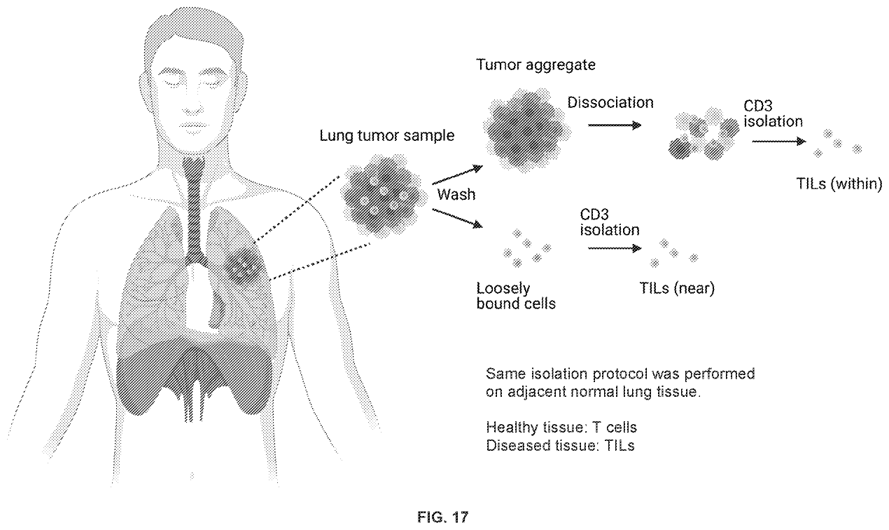

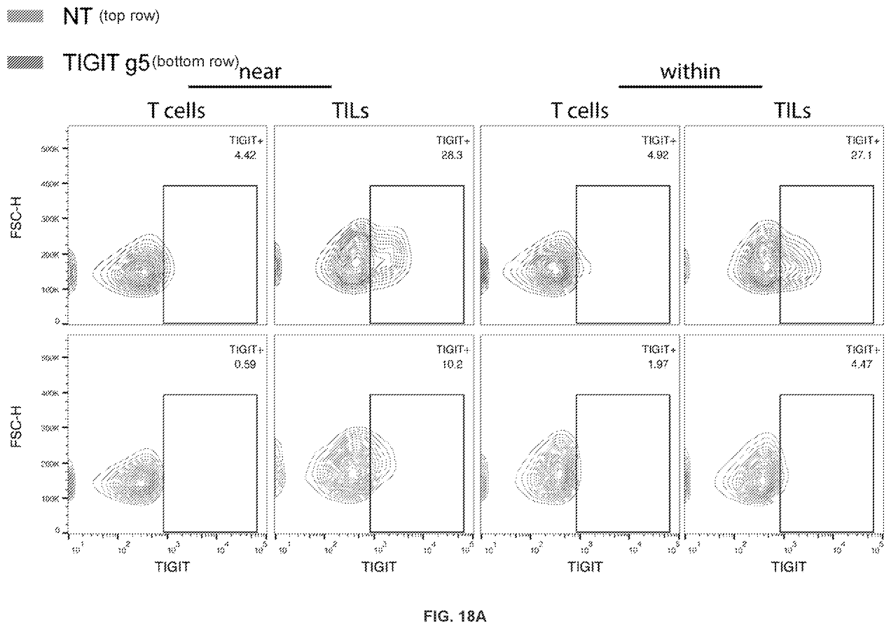

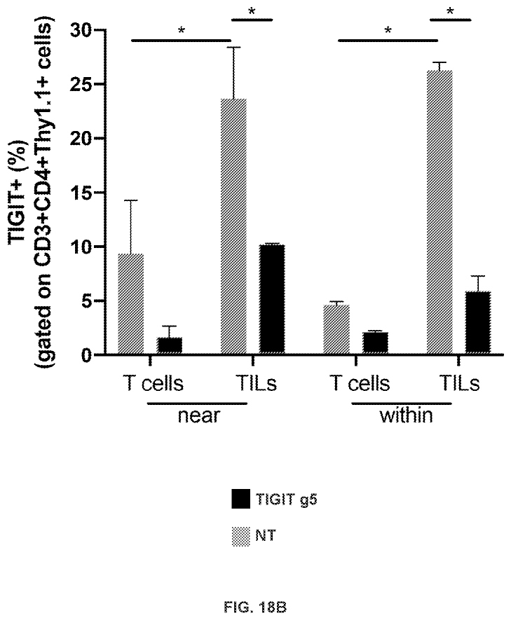

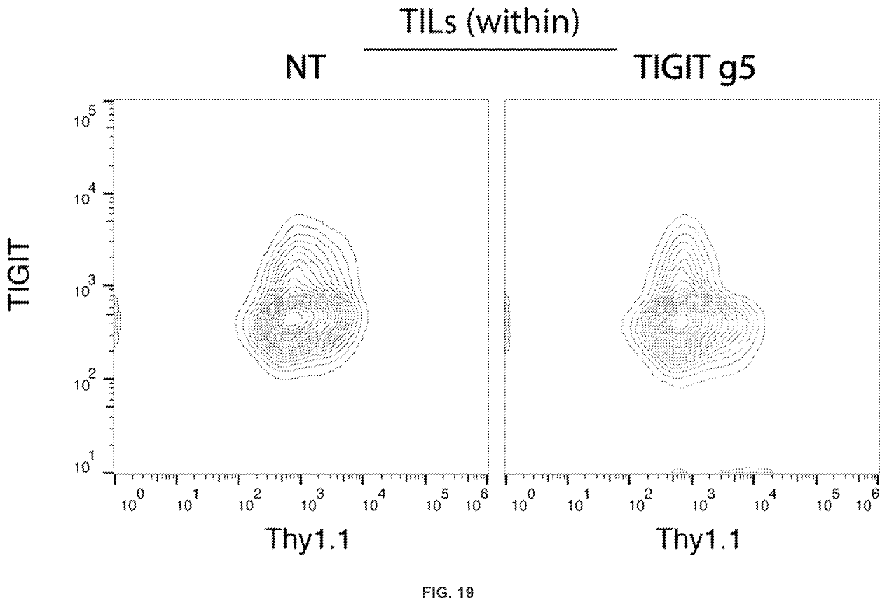

It was tested whether the epigenome technologies (described in Examples 2-4) could function in clinically relevant T cells such as tumor-infiltrating lymphocytes (TILs), which are often confined to an exhausted state (FIG. 17). The gRNAs used to target TIGIT are shown in TABLE 1 above. Robust proliferation, transduction, and target gene repression were observed in both freshly isolated TILs and TILs expanded for several weeks in media supplemented with high concentrations of IL-2 (FIG. 16A, FIG. 16B). Specifically, expression of TIGIT, a clinically relevant checkpoint surface marker expressed in >25% of cells, was repressed to <5% in freshly isolated TILs. TIGIT gRNA5 was particularly effective (FIG. 18A, FIG. 18B). TIGIT gene repression was dependent on the presence of SaCas9 and the gRNA (FIG. 19).

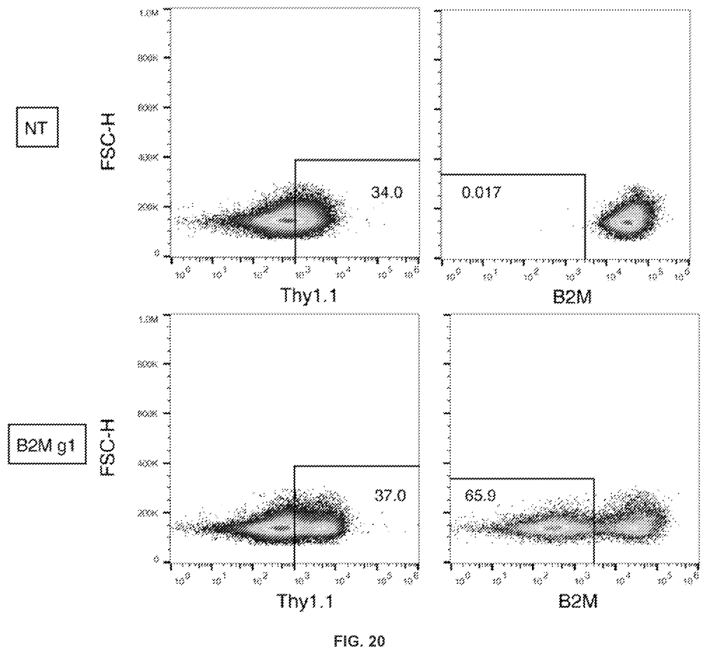

The most potent B2M gRNA (B2M gRNA1, H1) was tested in TILs that had been expanded for 2-3 weeks in high concentrations of IL-2. Greater than 35% transduction rates were observed with >65% of transduced cells silencing B2M (FIG. 20). Collectively, these data indicated the epigenomic tools could effectively function in T cells derived from healthy PBMC donors as well as from diseased patients.

### Example 6

### dSaCas9-Activators

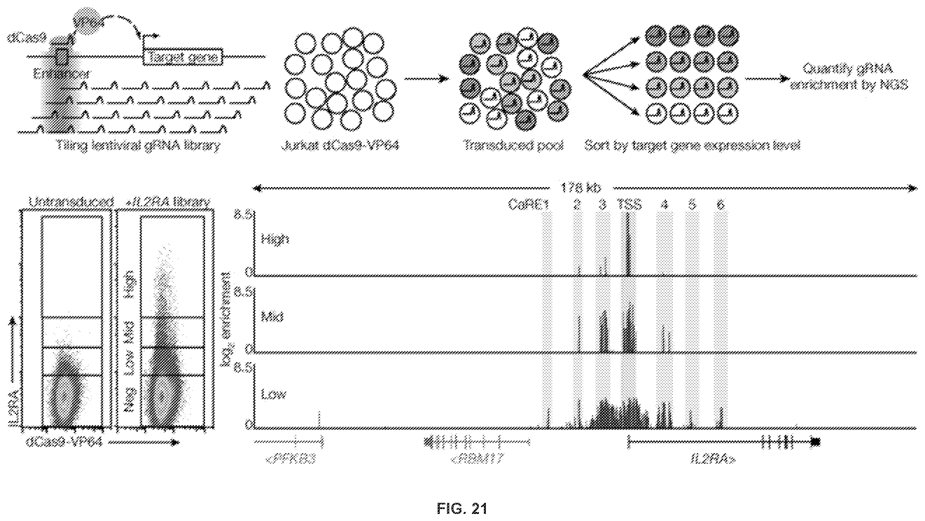

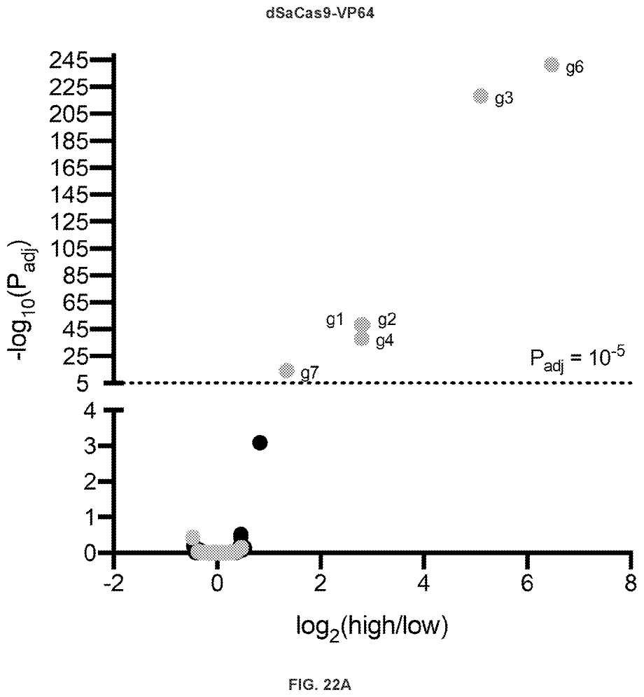

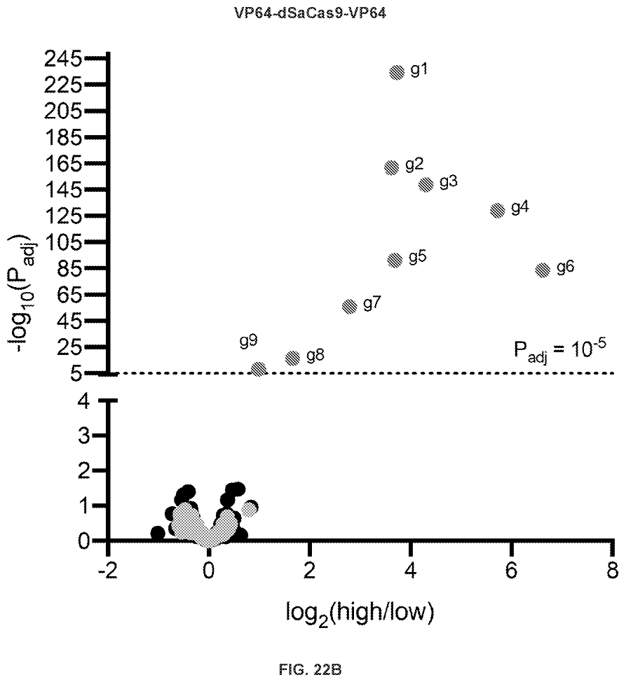

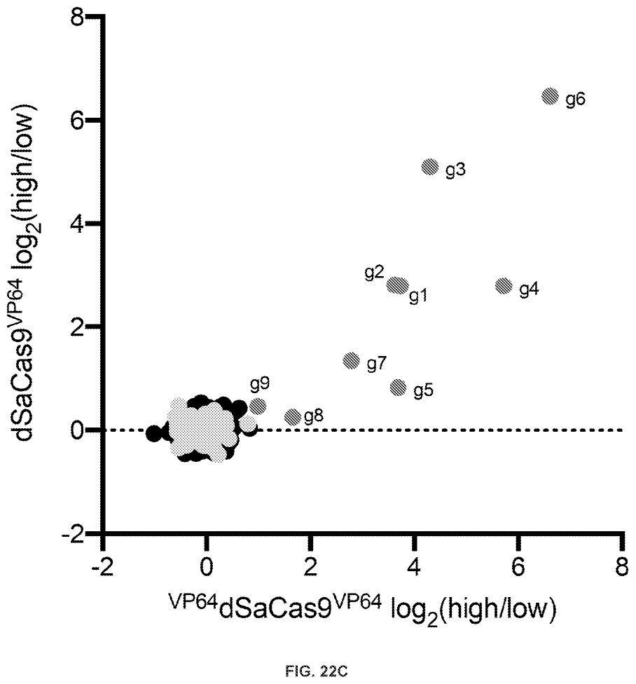

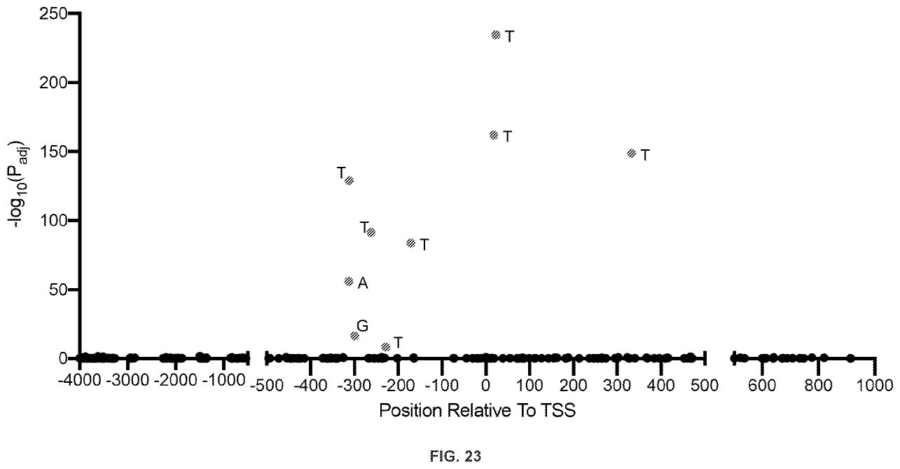

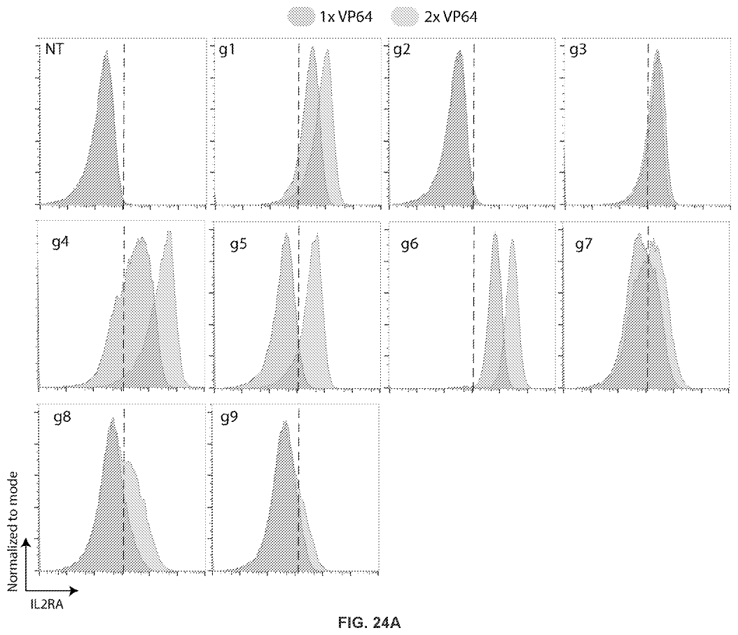

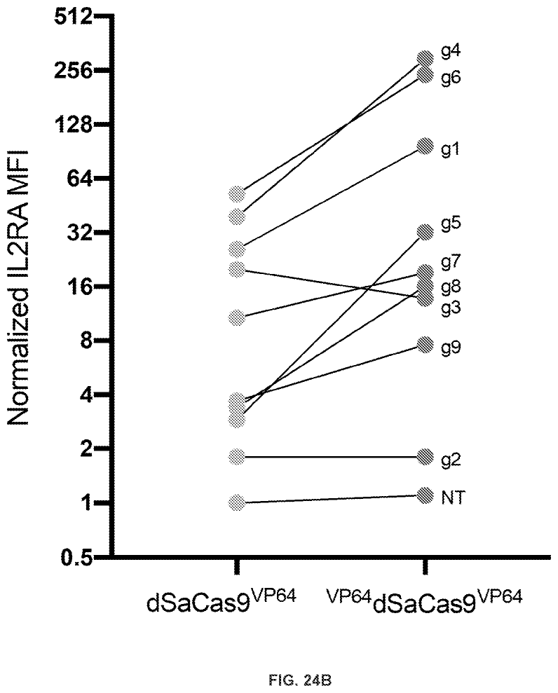

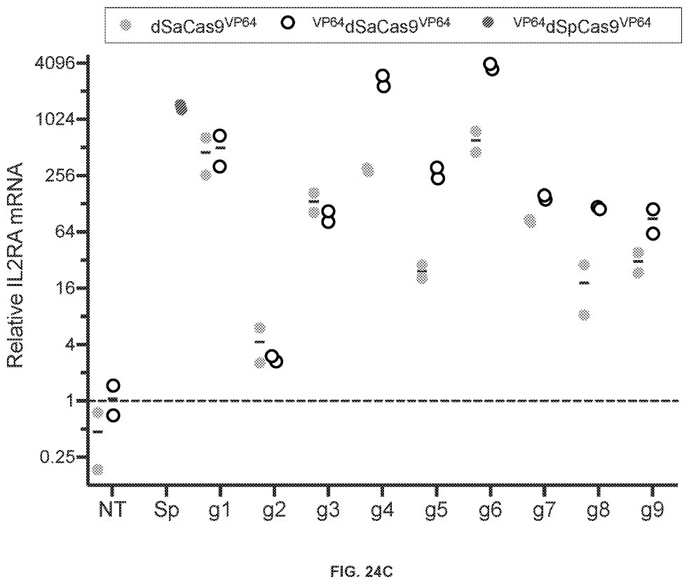

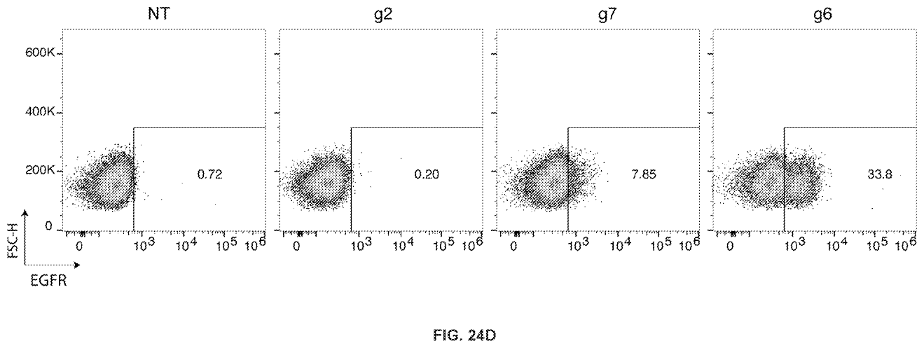

A small and robust dSaCas9 activator was developed to enable scalable CRISPRa screens in primary human T cells. While dSaCas9VP64 has achieved modest activation of target genes, an additional copy of VP64 (150 bp) was fused to its N-terminus to see if it improved function without compromising lentiviral production. dSaCas9VP64 and VP64dSaCas9VP64 were compared by conducting parallel CRISPRa screens in stable polyclonal Jurkat lines using a gRNA library tiling a 10 kb window around the IL2RA promoter (FIG. 21). The gRNAs used to target IL2RA are shown in TABLE 1. More gRNA hits and a marked increase in IL2RA activation was observed with VP64dSaCas9VP64 relative to dSaCas9VP64 (FIG. 22A, FIG. 22B, FIG. 22C, FIG. 24A, FIG. 24B), and activation was on par with the most potent dSpCas9 activator known (FIG. 24C). Most of the dSaCas9 gRNA hits fell within 300 bp upstream of the TSS, and all the hits were within 500 bp of the TSS, consistent with previous work defining parameters for optimized SpCas9 gRNA activation libraries (FIG. 23).

Further. VP64dSaCas9VP64 was used to upregulate EGFR in primary human T cells, which confirmed that VP64dSaCas9VP64 could be used to upregulate endogenous genes in primary human T cells (FIG. 24D). These data identified a potent activator for CRISPRa screens in human T cells.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A CRISPR/Cas system comprising: a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, and/or DNA demethylase activity; and at least one guide RNA (gRNA) targeting a gene or a regulatory element thereof in an immune cell.

Clause 2. A CRISPR/Cas system comprising: a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, and/or DNA demethylase activity; and at least one guide RNA (gRNA) targeting a gene selected from B2M, TIGIT, CD2, EGFR, and IL2RA, or a regulatory element thereof in a cell.

Clause 3. The CRISPR/Cas system of clause 2, wherein the cell is an immune cell.

Clause 4. The CRISPR/Cas system of clause 1 or 3, wherein the immune cell is a T cell.

Clause 5. The CRISPR/Cas system of any one of clauses 1-4, wherein the first polypeptide domain comprises a Cas9 protein.

Clause 6. The CRISPR/Cas system of clause 5, wherein the first polypeptide domain comprises a Staphylococcus aureus Cas9 protein (SaCas9).

Clause 7. The CRISPR/Cas system of clause 5, wherein the first polypeptide domain comprises a nuclease-inactivated Cas9 protein (dCas9).

Clause 8. The CRISPR/Cas system of clause 6 or 7, wherein the first polypeptide domain comprises a nuclease-inactivated Staphylococcus aureus Cas9 protein (dSaCas9).

Clause 9. The CRISPR/Cas system of any one of clauses 1 and 4-8, wherein the gRNA targets a gene selected from B2M, TIGIT, CD2, EGFR, and IL2RA, or a regulatory element thereof.

Clause 10. The CRISPR/Cas system of clause 7, wherein the gRNA comprises a polynucleotide sequence selected from SEQ ID NOs: 58-70 or 102-120, a variant thereof, or a fragment thereof, or is encoded by or targets a polynucleotide sequence selected from SEQ ID NOs: 45-57 or 83-101.

Clause 11. The CRISPR/Cas system of clause 9 or 10, wherein the gRNA targets B2M or a regulatory element thereof.

Clause 12. The CRISPR/Cas system of clause 11, wherein the gRNA targets a sequence within 500 base pairs of the transcriptional start site of B2M.

Clause 13. The CRISPR/Cas system of clause 11 or 12, wherein the gRNA comprises a polynucleotide sequence selected from SEQ ID NOs: 68-70, a variant thereof, or a fragment thereof.

Clause 14. The CRISPR/Cas system of clause 11 or 12, wherein the gRNA is encoded by or targets a polynucleotide comprising a sequence selected from SEQ ID NOs: 53-57.

Clause 15. The CRISPR/Cas system of clause 9 or 10, wherein the gRNA targets TIGIT or a regulatory element thereof.

Clause 16. The CRISPR/Cas system of clause 15, wherein the gRNA targets a sequence within 500 base pairs of the transcriptional start site of TIGIT.

Clause 17. The CRISPR/Cas system of clause 15 or 16, wherein the gRNA comprises the polynucleotide sequence of SEQ ID NO: 110, a variant thereof, or a fragment thereof.

Clause 18. The CRISPR/Cas system of clause 15 or 16, wherein the gRNA is encoded by or targets a polynucleotide comprising the sequence of SEQ ID NO: 91.

Clause 19. The CRISPR/Cas system of clause 9 or 10, wherein the gRNA targets CD2 or a regulatory element thereof.

Clause 20. The CRISPR/Cas system of clause 19, wherein the gRNA targets a sequence within 500 base pairs of the transcriptional start site of CD2.

Clause 21. The CRISPR/Cas system of clause 19 or 20, wherein the gRNA comprises a polynucleotide sequence selected from SEQ ID NOs: 58-65 or 102-109, a variant thereof, or a fragment thereof.

Clause 22. The CRISPR/Cas system of clause 19 or 20, wherein the gRNA is encoded by or targets a polynucleotide comprising a sequence selected from SEQ ID NOs: 45-52 or 83-90.

Clause 23. The CRISPR/Cas system of clause 9 or 10, wherein the gRNA targets EGFR or a regulatory element thereof.

Clause 24. The CRISPR/Cas system of clause 23, wherein the gRNA targets a sequence within 500 base pairs of the transcriptional start site of EGFR.

Clause 25. The CRISPR/Cas system of clause 23 or 24, wherein the gRNA comprises the polynucleotide sequence of SEQ ID NO: 101, a variant thereof, or a fragment thereof.

Clause 26. The CRISPR/Cas system of clause 23 or 24, wherein the gRNA is encoded by or targets a polynucleotide comprising the sequence of SEQ ID NO: 120.

Clause 27. The CRISPR/Cas system of clause 9 or 10, wherein the gRNA targets IL2RA or a regulatory element thereof.

Clause 28. The CRISPR/Cas system of clause 27, wherein the gRNA targets a sequence within 500 base pairs of the transcriptional start site of IL2RA.

Clause 29. The CRISPR/Cas system of clause 27 or 28, wherein the gRNA comprises a polynucleotide sequence selected from SEQ ID NOs: 111-119, a variant thereof, or a fragment thereof.

Clause 30. The CRISPR/Cas system of clause 27 or 28, wherein the gRNA is encoded by or targets a polynucleotide comprising a sequence selected from SEQ ID NOs: 92-100.

Clause 31. The CRISPR/Cas system of any one of clauses 10-26, wherein the gRNA further comprises the polynucleotide sequence of SEQ ID NO: 19 or 126.

Clause 32. The CRISPR/Cas system of any one of clauses 1-31, wherein the second polypeptide domain has transcription repression activity.

Clause 33. The CRISPR/Cas system of clause 32, wherein the at least one guide RNA (gRNA) targets a gene selected from B2M, TIGIT, and CD2, or a regulatory element thereof.

Clause 34. The CRISPR/Cas system of clause 32 or 33, wherein the second polypeptide domain comprises a KRAB domain, EED domain, MECP2 domain, ERF repressor domain, Mxi1 repressor domain, SID4X repressor domain, Mad-SID repressor domain, DNMT3A or DNMT3L or fusion thereof, LSD1 histone demethylase, or TATA box binding protein domain.

Clause 35. The CRISPR/Cas system of clause 34, wherein the fusion protein comprises dSaCas9-KRAB.

Clause 36. The CRISPR/Cas system of any one of clauses 1-31, wherein the second polypeptide domain has transcription activation activity.

Clause 37. The CRISPR/Cas system of clause 36, wherein the at least one guide RNA (gRNA) targets a gene selected from CD2, EGFR, and IL2RA, or a regulatory element thereof.

Clause 38. The CRISPR/Cas system of clause 36 or 37, wherein the second polypeptide domain comprises a VP16, a VP48, a VP64, a p65, a TET1, a VPR, a VPH, a Rta, or a p300 protein, or a fragment thereof or a combination thereof.

Clause 39. The CRISPR/Cas system of clause 38, wherein the fusion protein comprises dSaCas9-VP64, VP64-dSaCas9-VP64, or dSaCas9-p300^{core}.

Clause 40. An isolated polynucleotide encoding the CRISPR/Cas system of any one of clauses 1-39.

Clause 41. A vector comprising the isolated polynucleotide of clause 40.

Clause 42. A cell comprising the isolated polynucleotide of clause 40 or the vector of clause 41.

Clause 43. A vector composition comprising: a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and a polynucleotide sequence encoding at least one guide RNA (gRNA) targeting a gene or a regulatory element thereof in an immune cell.

Clause 44. A vector composition comprising: a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and a polynucleotide sequence encoding at least one guide RNA (gRNA) targeting a gene selected from B2M, TIGIT, CD2, EGFR, and IL2RA, or a regulatory element thereof in a cell.

Clause 45. The vector composition of clause 44, wherein the cell is an immune cell.

Clause 46. The vector composition of clause 43 or 45, wherein the immune cell is a T cell.

Clause 47. The vector composition of any one of clauses 43-46, wherein the first polypeptide domain comprises a Cas9 protein.

Clause 48. The vector composition of clause 47, wherein the first polypeptide domain comprises a Staphylococcus aureus Cas9 protein (SaCas9).

Clause 49. The vector composition of clause 47, wherein the first polypeptide domain comprises a nuclease-inactivated Cas9 protein (dCas9).

Clause 50. The vector composition of clause 48 or 49, wherein the first polypeptide domain comprises a nuclease-inactivated Staphylococcus aureus Cas9 protein (dSaCas9).

Clause 51. The vector composition of any one of clauses 43-50, wherein the vector composition comprises a first vector comprising the polynucleotide sequence encoding a fusion protein, and a second vector comprising the polynucleotide sequence encoding at least one gRNA.

Clause 52. The vector composition of any one of clauses 43-50, wherein the vector composition comprises a single vector comprising the polynucleotide sequence encoding a fusion protein and the polynucleotide sequence encoding the at least one gRNA.

Clause 53. The vector composition of any one of clauses 43-52, further comprising a polynucleotide sequence encoding a reporter protein operably linked to the polynucleotide sequence encoding the fusion protein.

Clause 54. The vector composition of clause 53, wherein the reporter protein comprises a fluorescent protein and/or a protein detectable with an antibody.

Clause 55. The vector composition of clause 53 or 54, further comprising a polynucleotide sequence encoding a 2A self-cleaving peptide operably linked to the polynucleotide sequence encoding the fusion protein and to the polynucleotide sequence encoding the reporter protein, wherein the T2A polynucleotide sequence is between the polynucleotide sequence encoding the fusion protein and the polynucleotide sequence encoding the reporter protein.

Clause 56. The vector composition of any one of clauses 43-55, wherein the gRNA targets a gene selected from B2M, TIGIT, CD2, EGFR, and IL2RA, or a regulatory element thereof.

Clause 57. The vector composition of clause 56, wherein the gRNA comprises a polynucleotide sequence selected from SEQ ID NOs: 58-70 or 102-120, a variant thereof, or a fragment thereof, or is encoded by or targets a polynucleotide sequence selected from SEQ ID NOs: 45-57 or 83-101.

Clause 58. The vector composition of clause 56 or 57, wherein the gRNA targets B2M or a regulatory element thereof.

Clause 59. The vector composition of clause 58, wherein the gRNA targets a sequence within 500 base pairs of the transcriptional start site of B2M.

Clause 60. The vector composition of clause 58 or 59, wherein the gRNA comprises a polynucleotide sequence selected from SEQ ID NOs: 66-70, a variant thereof, or a fragment thereof.

Clause 61. The vector composition of clause 58 or 59, wherein the gRNA is encoded by or targets a polynucleotide sequence selected from SEQ ID NOs: 53-57.

Clause 62. The vector composition of clause 56 or 57, wherein the gRNA targets TIGIT or a regulatory element thereof.

Clause 63. The vector composition of clause 62, wherein the gRNA targets a sequence within 500 base pairs of the transcriptional start site of TIGIT.

Clause 64. The vector composition of clause 62 or 63, wherein the gRNA comprises the polynucleotide sequence of SEQ ID NO: 110, a variant thereof, or a fragment thereof.

Clause 65. The vector composition of clause 62 or 63, wherein the gRNA is encoded by or targets a polynucleotide comprising the sequence of SEQ ID NO: 91.

Clause 66. The vector composition of clause 56 or 57, wherein the gRNA targets CD2 or a regulatory element thereof.

Clause 67. The vector composition of clause 66, wherein the gRNA targets a sequence within 500 base pairs of the transcriptional start site of CD2.

Clause 68. The vector composition of clause 66 or 67, wherein the gRNA comprises a polynucleotide sequence selected from SEQ ID NOs: 58-65 or 102-109, a variant thereof, or a fragment thereof.

Clause 69. The vector composition of clause 66 or 67, wherein the gRNA is encoded by or targets a polynucleotide comprising a sequence selected from SEQ ID NOs: 45-52 or 83-90.

Clause 70. The vector composition of clause 56 or 57, wherein the gRNA targets EGFR or a regulatory element thereof.

Clause 71. The vector composition of clause 70, wherein the gRNA targets a sequence within 500 base pairs of the transcriptional start site of EGFR.

Clause 72. The vector composition of clause 70 or 71, wherein the gRNA comprises the polynucleotide sequence of SEQ ID NO: 120, a variant thereof, or a fragment thereof.

Clause 73. The vector composition of clause 70 or 71, wherein the gRNA is encoded by or targets a polynucleotide comprising the sequence of SEQ ID NO: 101.

Clause 74. The vector composition of clause 56 or 57, wherein the gRNA targets IL2RA or a regulatory element thereof.

Clause 75. The vector composition of clause 74, wherein the gRNA targets a sequence within 500 base pairs of the transcriptional start site of IL2RA.

Clause 76. The vector composition of clause 74 or 75, wherein the gRNA comprises a polynucleotide sequence selected from SEQ ID NOs: 111-119, a variant thereof, or a fragment thereof.

Clause 77. The vector composition of clause 74 or 75, wherein the gRNA is encoded by or targets a polynucleotide sequence selected from SEQ ID NOs: 92-100.

Clause 78. The vector composition of any one of clauses 57-77, wherein the gRNA further comprises the polynucleotide sequence of SEQ ID NO: 19 or 126.

Clause 79. The vector composition of any one of clauses 43-78, wherein the second polypeptide domain has transcription repression activity.

Clause 80. The vector composition of clause 79, wherein the at least one guide RNA (gRNA) targets a gene selected from B2M, TIGIT, and CD2, or a regulatory element thereof.

Clause 81. The vector composition of clause 79 or 80, wherein the second polypeptide domain comprises a KRAB domain, EED domain, MECP2 domain, DNMT3A or DNMT3L or fusion thereof, ERF repressor domain, Mxi1 repressor domain, SID4X repressor domain, Mad-SID repressor domain, LSD1 histone demethylase, or TATA box binding protein domain.

Clause 82. The vector composition of clause 81, wherein the fusion protein comprises dSaCas9-KRAB.

Clause 83. The vector composition of any one of clauses 43-78, wherein the second polypeptide domain has transcription activation activity.

Clause 84. The vector composition of clause 83, wherein the at least one guide RNA (gRNA) targets a gene selected from CD2, EGFR, and IL2RA, or a regulatory element thereof.

Clause 85. The vector composition of clause 83 or 84, wherein the second polypeptide domain comprises a VP16, a VP48, a VP64, a p65, a TET1, a VPR, a VPH, a Rta, or a p300 protein, or a fragment thereof or a combination thereof.

Clause 86. The vector composition of clause 85, wherein the fusion protein comprises dSaCas9-VP64, VP64-dSaCas9-VP64, or dSaCas9-p300^{core}.

Clause 87. The vector composition of any one of clauses 43-86, further comprising a human Pol III U6 promoter upstream of and driving expression of the polynucleotide sequence encoding the gRNA, wherein the human Pol III U6 promoter and the polynucleotide sequence encoding the gRNA are orientated in the opposite direction from the polynucleotide sequence encoding the fusion protein.

Clause 88. The vector composition of any one of clauses 43-87, wherein the vector composition comprises a lentiviral vector comprising the polynucleotide sequence encoding a fusion protein and/or the polynucleotide sequence encoding the gRNA.

Clause 89. A method of modulating expression of a gene in a cell, the method comprising administering to the cell the CRISPR/Cas system of any one of clauses 1-39, the isolated polynucleotide of clause 40, the vector of clause 41, or the vector composition of any one of clauses 43-88.

Clause 90. A method of reducing B2M expression in a cell, the method comprising administering to the cell the CRISPR/Cas system of any one of clauses 1-14 or 31-35, the isolated polynucleotide of clause 40, the vector of clause 41, or the vector composition of any one of clauses 43-61, 78-82, or 87-88.

Clause 91. A method of reducing immunological activity of a cell, the method comprising administering to the cell the CRISPR/Cas system of any one of clauses 1-14 or 31-35, the isolated polynucleotide of clause 40, the vector of clause 41, or the vector composition of any one of clauses 43-61, 78-82, or 87-88.

Clause 92. A method of reducing TIGIT expression in a cell, the method comprising administering to the cell the CRISPR/Cas system of any one of clauses 1-10, 15-18, or 31-35, the isolated polynucleotide of clause 40, the vector of clause 41, or the vector composition of any one of clauses 43-57, 62-65, 78-82, or 87-88.

Clause 93. A method of increasing an immune cell's ability to kill a cancer cell, the method comprising administering to the immune cell the CRISPR/Cas system of any one of clauses 1-10, 15-18, or 31-35, the isolated polynucleotide of clause 40, the vector of clause 41, or the vector composition of any one of clauses 43-57, 62-65, 78-82, or 87-88.

Clause 94. A method of reducing CD2 expression in a cell, the method comprising administering to the cell the CRISPR/Cas system of any one of clauses 1-10, 19-22, or 31-35, the isolated polynucleotide of clause 40, the vector of clause 41, or the vector composition of any one of clauses 43-57, 66-69, 78-82, or 87-88.

Clause 95. A method of increasing CD2 expression in a cell, the method comprising administering to the cell the CRISPR/Cas system of any one of clauses 1-10, 19-22, 31, or 36-39, the isolated polynucleotide of clause 40, the vector of clause 41, or the vector composition of any one of clauses 43-57, 66-69, 78, or 83-88.

Clause 96. A method of increasing EGFR expression in a cell, the method comprising administering to the cell the CRISPR/Cas system of any one of clauses 1-10, 23-26, 31, or 36-39, the isolated polynucleotide of clause 40, the vector of clause 41, or the vector composition of any one of clauses 43-57, 70-73, 78, or 83-88.

Clause 97. A method of increasing IL2RA expression in a cell, the method comprising administering to the cell the CRISPR/Cas system of any one of clauses 1-10, 27-31, or 36-39, the isolated polynucleotide of clause 40, the vector of clause 41, or the vector composition of any one of clauses 43-57, 74-78, or 83-88.

Clause 98. The method of any one of clauses 89-97, wherein the cell is an immune cell.

Clause 99. The method of clause 98, wherein the immune cell is a T cell.

Clause 100. A cell modified by the method of any one of clauses 89-97.

Clause 101. A method of treating a subject having a disease, the method comprising administering to the subject the CRISPR/Cas system of any one of clauses 1-39, the isolated polynucleotide of clause 40, the vector of clause 41, the cell of clause 42, the vector composition of any one of clauses 43-88, or the cell of clause 100.

Clause 102. The method of clause 101, wherein the disease comprises cancer, an autoimmune disease, or a viral infection.

Clause 103. A method of screening for one or more putative gene regulatory elements in a genome that modulate a gene target or a phenotype of an immune cell, the method comprising: (a) contacting a plurality of modified target immune cells with a library of gRNAs, each gRNA targeting a gene regulatory element in an immune cell, thereby generating a pool of test immune cells, (b) selecting a population of test immune cells having a modulated gene or phenotype; (c) quantifying the frequency of the gRNAs within the population of selected immune cells, wherein the gRNAs that target gene regulatory elements that modulate the phenotype are overrepresented or underrepresented in the selected immune cells; and (d) identifying and characterizing the gRNAs within the population of selected immune cells thereby identifying the gene regulatory elements that modulate the phenotype, wherein the modified target immune cell comprises a fusion protein, the fusion protein comprising a first polypeptide domain comprising a Cas protein and a second polypeptide domain having an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity.

Clause 104. The method of clause 103, wherein the immune cell is a T cell.

Clause 105. The method of any one of clauses 103-104, wherein the first polypeptide domain comprises a Cas9 protein.

Clause 106. The method of clause 105, wherein the first polypeptide domain comprises a Staphylococcus aureus Cas9 protein (SaCas9).

Clause 107. The method of clause 106, wherein the first polypeptide domain comprises a nuclease-inactivated Staphylococcus aureus Cas9 protein (dSaCas9).

Clause 108. A method of screening a library of gRNAs for modulation of gene expression in a cell, the method comprising: (a) generating a library of vectors with a library of gRNAs, each gRNA targeting a target gene or a regulatory element thereof in a cell, the library of vectors comprising: a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; a polynucleotide sequence encoding a reporter protein operably linked to the polynucleotide sequence encoding the fusion protein; and a polynucleotide sequence encoding one of the gRNAs; (b) transducing a plurality of cells with the library of gRNAs; (c) culturing the transduced cells; (d) sorting the cultured cells based on the growth of the cells or on the level of expression of the gene or the reporter protein; and (e) sequencing the gRNA from each cell sorted in step (d).

Clause 109. The method of clause 108, wherein the reporter protein comprises a fluorescent protein and/or a protein detectable with an antibody, and wherein the cultured cells are sorted in step (d) based on the level of expression of the reporter protein.

Clause 110. The method of clause 108 or 109, wherein the cell is an immune cell.

Clause 111. The method of clause 110, wherein the immune cell is a T cell.

Clause 112. The method of any one of clauses 108-111, wherein the first polypeptide domain comprises a Cas9 protein.

Clause 113. The method of clause 112, wherein the first polypeptide domain comprises a Staphylococcus aureus Cas9 protein (SaCas9).

Clause 114. The method of clause 113, wherein the first polypeptide domain comprises a nuclease-inactivated Staphylococcus aureus Cas9 protein (dSaCas9).

Clause 115. The method of any one of clauses 108-114, wherein the library of vectors further comprises a polynucleotide sequence encoding a 2A self-cleaving peptide operably linked to the polynucleotide sequence encoding the fusion protein and to the polynucleotide sequence encoding the reporter protein, wherein the polynucleotide sequence encoding a 2A self-cleaving peptide is between the polynucleotide sequence encoding the fusion protein and the polynucleotide sequence encoding the reporter protein.

Clause 116. The method of any one of clauses 108-115, further comprising: (f) identifying the target gene of the gRNA sequenced in step (e).

Clause 117. The method of clause 116, further comprising: (g) modulating the level of the gene target discovered in (f) or modulating the activity of the protein produced from the gene target discovered in (f) for enhancing properties of a cell therapy.

Clause 118. A method of screening a library of gRNAs for modulation of gene expression in a cell, the method comprising: (a) generating a library of vectors with a library of gRNAs, each gRNA targeting a target gene or a regulatory element thereof in a cell, the library of vectors comprising: a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and a polynucleotide sequence encoding one of the gRNAs; (b) transducing a plurality of cells with the library of gRNAs; (c) culturing the transduced cells; (d) capturing the gRNA from the transduced cells; and (e) sequencing the gRNA from each transduced cell captured in step (d).

Clause 119. The method of clause 118, wherein the gRNA from the transduced cells is captured with single cell technology in step (d).

Clause 120. The method of clause 118 or 119, wherein the method further comprises determining the level of mRNA expression and/or the level of protein expression in the transduced cells.

Clause 121. The method of clause 120, wherein the method further comprises: grouping transduced cells having the same gRNA; and comparing the target gene expression of transduced cells having the same gRNA, at the mRNA and/or protein level, to the target gene expression of cells without the same gRNA.

Clause 122. The method of any one of clauses 118-121, further comprising identifying the target gene of the gRNA sequenced in step (e).

Clause 123. The method of clause 122, further comprising modulating the level of the gene target or modulating the activity of the protein produced from the gene target for enhancing properties of a cell therapy.

Clause 124. The method of any one of clauses 118-123, wherein the cell is an immune cell.

Clause 125. The method of clause 124, wherein the immune cell is a T cell.

Clause 126. The method of any one of clauses 118-125, wherein the first polypeptide domain comprises a Staphylococcus aureus Cas9 protein (SaCas9).

Clause 127. The method of clause 128, wherein the first polypeptide domain comprises a nuclease-inactivated Staphylococcus aureus Cas9 protein (dSaCas9).

1. A CRISPR/Cas system comprising:
a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, and/or DNA demethylase activity; and
at least one guide RNA (gRNA) targeting a gene or a regulatory element thereof in an immune cell.

2. A CRISPR/Cas system comprising:
a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, and/or DNA demethylase activity; and
at least one guide RNA (gRNA) targeting a gene selected from B2M, TIGIT, CD2, EGFR, and IL2RA, or a regulatory element thereof in a cell.

3. The CRISPR/Cas system of claim 2, wherein the cell is an immune cell.

4. The CRISPR/Cas system of claim 1 or 3, wherein the immune cell is a T cell.

5. The CRISPR/Cas system of any one of claims 1-4, wherein the first polypeptide domain comprises a Cas9 protein.

6. The CRISPR/Cas system of claim 5, wherein the first polypeptide domain comprises a Staphylococcus aureus Cas9 protein (SaCas9).

7. The CRISPR/Cas system of claim 5, wherein the first polypeptide domain comprises a nuclease-inactivated Cas9 protein (dCas9).

8. The CRISPR/Cas system of claim 6 or 7, wherein the first polypeptide domain comprises a nuclease-inactivated Staphylococcus aureus Cas9 protein (dSaCas9).

9. The CRISPR/Cas system of any one of claims 1 and 4-8, wherein the gRNA targets a gene selected from B2M, TIGIT, CD2, EGFR, and IL2RA, or a regulatory element thereof.

10. The CRISPR/Cas system of claim 7, wherein the gRNA comprises a polynucleotide sequence selected from SEQ ID NOs: 58-70 or 102-120, a variant thereof, or a fragment thereof, or is encoded by or targets a polynucleotide sequence selected from SEQ ID NOs: 45-57 or 83-101.

11. The CRISPR/Cas system of claim 9 or 10, wherein the gRNA targets B2M or a regulatory element thereof.

12. The CRISPR/Cas system of claim 11, wherein the gRNA targets a sequence within 500 base pairs of the transcriptional start site of B2M.

13. The CRISPR/Cas system of claim 11 or 12, wherein the gRNA comprises a polynucleotide sequence selected from SEQ ID NOs: 66-70, a variant thereof, or a fragment thereof.

14. The CRISPR/Cas system of claim 11 or 12, wherein the gRNA is encoded by or targets a polynucleotide comprising a sequence selected from SEQ ID NOs: 53-57.

15. The CRISPR/Cas system of claim 9 or 10, wherein the gRNA targets TIGIT or a regulatory element thereof.

16. The CRISPR/Cas system of claim 15, wherein the gRNA targets a sequence within 500 base pairs of the transcriptional start site of TIGIT.

17. The CRISPR/Cas system of claim 15 or 16, wherein the gRNA comprises the polynucleotide sequence of SEQ ID NO: 110, a variant thereof, or a fragment thereof.

18. The CRISPR/Cas system of claim 15 or 16, wherein the gRNA is encoded by or targets a polynucleotide comprising the sequence of SEQ ID NO: 91.

19. The CRISPR/Cas system of claim 9 or 10, wherein the gRNA targets CD2 or a regulatory element thereof.

20. The CRISPR/Cas system of claim 19, wherein the gRNA targets a sequence within 500 base pairs of the transcriptional start site of CD2.

21. The CRISPR/Cas system of claim 19 or 20, wherein the gRNA comprises a polynucleotide sequence selected from SEQ ID NOs: 58-65 or 102-109, a variant thereof, or a fragment thereof.

22. The CRISPR/Cas system of claim 19 or 20, wherein the gRNA is encoded by or targets a polynucleotide comprising a sequence selected from SEQ ID NOs: 45-52 or 83-90.

23. The CRISPR/Cas system of claim 9 or 10, wherein the gRNA targets EGFR or a regulatory element thereof.

24. The CRISPR/Cas system of claim 23, wherein the gRNA targets a sequence within 500 base pairs of the transcriptional start site of EGFR.

25. The CRISPR/Cas system of claim 23 or 24, wherein the gRNA comprises the polynucleotide sequence of SEQ ID NO: 101, a variant thereof, or a fragment thereof.

26. The CRISPR/Cas system of claim 23 or 24, wherein the gRNA is encoded by or targets a polynucleotide comprising the sequence of SEQ ID NO: 120.

27. The CRISPR/Cas system of claim 9 or 10, wherein the gRNA targets IL2RA or a regulatory element thereof.

28. The CRISPR/Cas system of claim 27, wherein the gRNA targets a sequence within 500 base pairs of the transcriptional start site of IL2RA.

29. The CRISPR/Cas system of claim 27 or 28, wherein the gRNA comprises a polynucleotide sequence selected from SEQ ID NOs: 111-119, a variant thereof, or a fragment thereof.

30. The CRISPR/Cas system of claim 27 or 28, wherein the gRNA is encoded by or targets a polynucleotide comprising a sequence selected from SEQ ID NOs: 92-100.

31. The CRISPR/Cas system of any one of claims 10-26, wherein the gRNA further comprises the polynucleotide sequence of SEQ ID NO: 19 or 126.

32. The CRISPR/Cas system of any one of claims 1-31, wherein the second polypeptide domain has transcription repression activity.

33. The CRISPR/Cas system of claim 32, wherein the at least one guide RNA (gRNA) targets a gene selected from B2M, TIGIT, and CD2, or a regulatory element thereof.

34. The CRISPR/Cas system of claim 32 or 33, wherein the second polypeptide domain comprises a KRAB domain, EED domain, MECP2 domain, ERF repressor domain, Mxi1 repressor domain, SID4X repressor domain, Mad-SID repressor domain, DNMT3A or DNMT3L or fusion thereof, LSD1 histone demethylase, or TATA box binding protein domain.

35. The CRISPR/Cas system of claim 34, wherein the fusion protein comprises dSaCas9-KRAB.

36. The CRISPR/Cas system of any one of claims 1-31, wherein the second polypeptide domain has transcription activation activity.

37. The CRISPR/Cas system of claim 36, wherein the at least one guide RNA (gRNA) targets a gene selected from CD2, EGFR, and IL2RA, or a regulatory element thereof.

38. The CRISPR/Cas system of claim 36 or 37, wherein the second polypeptide domain comprises a VP16, a VP48, a VP64, a p65, a TET1, a VPR, a VPH, a Rta, or a p300 protein, or a fragment thereof or a combination thereof.

39. The CRISPR/Cas system of claim 38, wherein the fusion protein comprises dSaCas9-VP64, VP64-dSaCas9-VP64, or dSaCas9-p300^{core}.

40. An isolated polynucleotide encoding the CRISPR/Cas system of any one of claims 1-39.

41. A vector comprising the isolated polynucleotide of claim 40.

42. A cell comprising the isolated polynucleotide of claim 40 or the vector of claim 41.

43. A vector composition comprising:
a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and
a polynucleotide sequence encoding at least one guide RNA (gRNA) targeting a gene or a regulatory element thereof in an immune cell.

44. A vector composition comprising:
a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and
a polynucleotide sequence encoding at least one guide RNA (gRNA) targeting a gene selected from B2M, TIGIT, CD2, EGFR, and IL2RA, or a regulatory element thereof in a cell.

45. The vector composition of claim 44, wherein the cell is an immune cell.

46. The vector composition of claim 43 or 45, wherein the immune cell is a T cell.

47. The vector composition of any one of claims 43-46, wherein the first polypeptide domain comprises a Cas9 protein.

48. The vector composition of claim 47, wherein the first polypeptide domain comprises a Staphylococcus aureus Cas9 protein (SaCas9).

49. The vector composition of claim 47, wherein the first polypeptide domain comprises a nuclease-inactivated Cas9 protein (dCas9).

50. The vector composition of claim 48 or 49, wherein the first polypeptide domain comprises a nuclease-inactivated Staphylococcus aureus Cas9 protein (dSaCas9).

51. The vector composition of any one of claims 43-50, wherein the vector composition comprises a first vector comprising the polynucleotide sequence encoding a fusion protein, and a second vector comprising the polynucleotide sequence encoding at least one gRNA.

52. The vector composition of any one of claims 43-50, wherein the vector composition comprises a single vector comprising the polynucleotide sequence encoding a fusion protein and the polynucleotide sequence encoding the at least one gRNA.

53. The vector composition of any one of claims 43-52, further comprising a polynucleotide sequence encoding a reporter protein operably linked to the polynucleotide sequence encoding the fusion protein.

54. The vector composition of claim 53, wherein the reporter protein comprises a fluorescent protein and/or a protein detectable with an antibody.

55. The vector composition of claim 53 or 54, further comprising a polynucleotide sequence encoding a 2A self-cleaving peptide operably linked to the polynucleotide sequence encoding the fusion protein and to the polynucleotide sequence encoding the reporter protein, wherein the T2A polynucleotide sequence is between the polynucleotide sequence encoding the fusion protein and the polynucleotide sequence encoding the reporter protein.

56. The vector composition of any one of claims 43-55, wherein the gRNA targets a gene selected from B2M, TIGIT, CD2, EGFR, and IL2RA, or a regulatory element thereof.

57. The vector composition of claim 58, wherein the gRNA comprises a polynucleotide sequence selected from SEQ ID NOs: 58-70 or 102-120, a variant thereof, or a fragment thereof, or is encoded by or targets a polynucleotide sequence selected from SEQ ID NOs: 45-57 or 83-101.

58. The vector composition of claim 58 or 57, wherein the gRNA targets B2M or a regulatory element thereof.

59. The vector composition of claim 58, wherein the gRNA targets a sequence within 500 base pairs of the transcriptional start site of B2M.

60. The vector composition of claim 58 or 59, wherein the gRNA comprises a polynucleotide sequence selected from SEQ ID NOs: 66-70, a variant thereof, or a fragment thereof.

61. The vector composition of claim 58 or 59, wherein the gRNA is encoded by or targets a polynucleotide sequence selected from SEQ ID NOs: 53-57.

62. The vector composition of claim 56 or 57, wherein the gRNA targets TIGIT or a regulatory element thereof.

63. The vector composition of claim 62, wherein the gRNA targets a sequence within 500 base pairs of the transcriptional start site of TIGIT.

64. The vector composition of claim 62 or 63, wherein the gRNA comprises the polynucleotide sequence of SEQ ID NO: 110, a variant thereof, or a fragment thereof.

65. The vector composition of claim 62 or 63, wherein the gRNA is encoded by or targets a polynucleotide comprising the sequence of SEQ ID NO: 91.

66. The vector composition of claim 56 or 57, wherein the gRNA targets CD2 or a regulatory element thereof.

67. The vector composition of claim 66, wherein the gRNA targets a sequence within 500 base pairs of the transcriptional start site of CD2.

68. The vector composition of claim 66 or 67, wherein the gRNA comprises a polynucleotide sequence selected from SEQ ID NOs: 58-65 or 102-109, a variant thereof, or a fragment thereof.

69. The vector composition of claim 66 or 67, wherein the gRNA is encoded by or targets a polynucleotide comprising a sequence selected from SEQ ID NOs: 45-52 or 83-90.

70. The vector composition of claim 56 or 57, wherein the gRNA targets EGFR or a regulatory element thereof.

71. The vector composition of claim 70, wherein the gRNA targets a sequence within 500 base pairs of the transcriptional start site of EGFR.

72. The vector composition of claim 70 or 71, wherein the gRNA comprises the polynucleotide sequence of SEQ ID NO: 120, a variant thereof, or a fragment thereof.

73. The vector composition of claim 70 or 71, wherein the gRNA is encoded by or targets a polynucleotide comprising the sequence of SEQ ID NO: 101.

74. The vector composition of claim 56 or 57, wherein the gRNA targets IL2RA or a regulatory element thereof.

75. The vector composition of claim 74, wherein the gRNA targets a sequence within 500 base pairs of the transcriptional start site of IL2RA.

76. The vector composition of claim 74 or 75, wherein the gRNA comprises a polynucleotide sequence selected from SEQ ID NOs: 111-119, a variant thereof, or a fragment thereof.

77. The vector composition of claim 74 or 75, wherein the gRNA is encoded by or targets a polynucleotide sequence selected from SEQ ID NOs: 92-100.

78. The vector composition of any one of claims 57-77, wherein the gRNA further comprises the polynucleotide sequence of SEQ ID NO: 19 or 126.

79. The vector composition of any one of claims 43-78, wherein the second polypeptide domain has transcription repression activity.

80. The vector composition of claim 79, wherein the at least one guide RNA (gRNA) targets a gene selected from B2M, TIGIT, and CD2, or a regulatory element thereof.

81. The vector composition of claim 79 or 80, wherein the second polypeptide domain comprises a KRAB domain, EED domain, MECP2 domain, DNMT3A or DNMT3L or fusion thereof, ERF repressor domain, Mxi1 repressor domain, SID4X repressor domain, Mad-SID repressor domain, LSD1 histone demethylase, or TATA box binding protein domain.

82. The vector composition of claim 81, wherein the fusion protein comprises dSaCas9-KRAB.

83. The vector composition of any one of claims 43-78, wherein the second polypeptide domain has transcription activation activity.

84. The vector composition of claim 83, wherein the at least one guide RNA (gRNA) targets a gene selected from CD2, EGFR, and IL2RA, or a regulatory element thereof.

85. The vector composition of claim 83 or 84, wherein the second polypeptide domain comprises a VP16, a VP48, a VP64, a p65, a TET1, a VPR, a VPH, a Rta, or a p300 protein, or a fragment thereof or a combination thereof.

86. The vector composition of claim 85, wherein the fusion protein comprises dSaCas9-VP64, VP64-dSaCas9-VP64, or dSaCas9-p300^{core}.

87. The vector composition of any one of claims 43-86, further comprising a human Pol Ill U6 promoter upstream of and driving expression of the polynucleotide sequence encoding the gRNA, wherein the human Pol III U6 promoter and the polynucleotide sequence encoding the gRNA are orientated in the opposite direction from the polynucleotide sequence encoding the fusion protein.

88. The vector composition of any one of claims 43-87, wherein the vector composition comprises a lentiviral vector comprising the polynucleotide sequence encoding a fusion protein and/or the polynucleotide sequence encoding the gRNA.

89. A method of modulating expression of a gene in a cell, the method comprising administering to the cell the CRISPR/Cas system of any one of claims 1-39, the isolated polynucleotide of claim 40, the vector of claim 41, or the vector composition of any one of claims 43-88.

90. A method of reducing B2M expression in a cell, the method comprising administering to the cell the CRISPR/Cas system of any one of claims 1-14 or 31-35, the isolated polynucleotide of claim 40, the vector of claim 41, or the vector composition of any one of claims 43-61, 78-82, or 87-88.

91. A method of reducing immunological activity of a cell, the method comprising administering to the cell the CRISPR/Cas system of any one of claims 1-14 or 31-35, the isolated polynucleotide of claim 40, the vector of claim 41, or the vector composition of any one of claims 43-81, 78-82, or 87-88.

92. A method of reducing TIGIT expression in a cell, the method comprising administering to the cell the CRISPR/Cas system of any one of claims 1-10, 15-18, or 31-35, the isolated polynucleotide of claim 40, the vector of claim 41, or the vector composition of any one of claims 43-57, 62-65, 78-82, or 87-88.

93. A method of increasing an immune cell's ability to kill a cancer cell, the method comprising administering to the immune cell the CRISPR/Cas system of any one of claims 1-10, 15-18, or 31-35, the isolated polynucleotide of claim 40, the vector of claim 41, or the vector composition of any one of claims 43-57, 62-65, 78-82, or 87-88.

94. A method of reducing CD2 expression in a cell, the method comprising administering to the cell the CRISPR/Cas system of any one of claims 1-10, 19-22, or 31-35, the isolated polynucleotide of claim 40, the vector of claim 41, or the vector composition of any one of claims 43-57, 66-69, 78-82, or 87-88.

95. A method of increasing CD2 expression in a cell, the method comprising administering to the cell the CRISPR/Cas system of any one of claims 1-10, 19-22, 31, or 36-39, the isolated polynucleotide of claim 40, the vector of claim 41, or the vector composition of any one of claims 43-57, 66-69, 78, or 83-88.

96. A method of increasing EGFR expression in a cell, the method comprising administering to the cell the CRISPR/Cas system of any one of claims 1-10, 23-26, 31, or 36-39, the isolated polynucleotide of claim 40, the vector of claim 41, or the vector composition of any one of claims 43-57, 70-73, 78, or 83-88.

97. A method of increasing IL2RA expression in a cell, the method comprising administering to the cell the CRISPR/Cas system of any one of claims 1-10, 27-31, or 38-39, the isolated polynucleotide of claim 40, the vector of claim 41, or the vector composition of any one of claims 43-57, 74-78, or 83-88.

98. The method of any one of claims 89-97, wherein the cell is an immune cell.

99. The method of claim 98, wherein the immune cell is a T cell.

100. A cell modified by the method of any one of claims 89-97.

101. A method of treating a subject having a disease, the method comprising administering to the subject the CRISPR/Cas system of any one of claims 1-39, the isolated polynucleotide of claim 40, the vector of claim 41, the cell of claim 42, the vector composition of any one of claims 43-88, or the cell of claim 100.

102. The method of claim 101, wherein the disease comprises cancer, an autoimmune disease, or a viral infection.

103. A method of screening for one or more putative gene regulatory elements in a genome that modulate a gene target or a phenotype of an immune cell, the method comprising:
(a) contacting a plurality of modified target immune cells with a library of gRNAs, each gRNA targeting a gene regulatory element in an immune cell, thereby generating a pool of test immune cells,
(b) selecting a population of test immune cells having a modulated gene or phenotype;
(c) quantifying the frequency of the gRNAs within the population of selected immune cells, wherein the gRNAs that target gene regulatory elements that modulate the phenotype are overrepresented or underrepresented in the selected immune cells; and
(d) identifying and characterizing the gRNAs within the population of selected immune cells thereby identifying the gene regulatory elements that modulate the phenotype,
wherein the modified target immune cell comprises a fusion protein, the fusion protein comprising a first polypeptide domain comprising a Cas protein and a second polypeptide domain having an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity.

104. The method of claim 103, wherein the immune cell is a T cell.

105. The method of any one of claims 103-104, wherein the first polypeptide domain comprises a Cas9 protein.

106. The method of claim 105, wherein the first polypeptide domain comprises a Staphylococcus aureus Cas9 protein (SaCas9).

107. The method of claim 106, wherein the first polypeptide domain comprises a nuclease-inactivated Staphylococcus aureus Cas9 protein (dSaCas9).

108. A method of screening a library of gRNAs for modulation of gene expression in a cell, the method comprising:
(a) generating a library of vectors with a library of gRNAs, each gRNA targeting a target gene or a regulatory element thereof in a cell, the library of vectors comprising:
a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity;
a polynucleotide sequence encoding a reporter protein operably linked to the polynucleotide sequence encoding the fusion protein; and
a polynucleotide sequence encoding one of the gRNAs;
(b) transducing a plurality of cells with the library of gRNAs;
(c) culturing the transduced cells;
(d) sorting the cultured cells based on the growth of the cells or on the level of expression of the gene or the reporter protein; and
(e) sequencing the gRNA from each cell sorted in step (d).

109. The method of claim 108, wherein the reporter protein comprises a fluorescent protein and/or a protein detectable with an antibody, and wherein the cultured cells are sorted in step (d) based on the level of expression of the reporter protein.

110. The method of claim 108 or 109, wherein the cell is an immune cell.

111. The method of claim 110, wherein the immune cell is a T cell.

112. The method of any one of claims 108-111, wherein the first polypeptide domain comprises a Cas9 protein.

113. The method of claim 112, wherein the first polypeptide domain comprises a Staphylococcus aureus Cas9 protein (SaCas9).

114. The method of claim 113, wherein the first polypeptide domain comprises a nuclease-inactivated Staphylococcus aureus Cas9 protein (dSaCas9).

115. The method of any one of claims 108-114, wherein the library of vectors further comprises a polynucleotide sequence encoding a 2A self-cleaving peptide operably linked to the polynucleotide sequence encoding the fusion protein and to the polynucleotide sequence encoding the reporter protein, wherein the polynucleotide sequence encoding a 2A self-cleaving peptide is between the polynucleotide sequence encoding the fusion protein and the polynucleotide sequence encoding the reporter protein.

116. The method of anyone of claims 108-115, further comprising:
(f) identifying the target gene of the gRNA sequenced in step (e).

117. The method of claim 116, further comprising:
(g) modulating the level of the gene target discovered in (f) or modulating the activity of the protein produced from the gene target discovered in (f) for enhancing properties of a cell therapy.

118. A method of screening a library of gRNAs for modulation of gene expression in a cell, the method comprising:
(a) generating a library of vectors with a library of gRNAs, each gRNA targeting a target gene or a regulatory element thereof in a cell, the library of vectors comprising:
a polynucleotide sequence encoding a fusion protein, wherein the fusion protein comprises two heterologous polypeptide domains, wherein the first polypeptide domain comprises a Cas protein, and wherein the second polypeptide domain has an activity selected from transcription activation activity, transcription repression activity, transcription release factor activity, histone modification activity, nuclease activity, nucleic acid association activity, histone methylase activity, DNA methylase activity, histone demethylase activity, or DNA demethylase activity; and
a polynucleotide sequence encoding one of the gRNAs;
(b) transducing a plurality of cells with the library of gRNAs;
(c) culturing the transduced cells;
(d) capturing the gRNA from the transduced cells; and
(e) sequencing the gRNA from each transduced cell captured in step (d).

119. The method of claim 118, wherein the gRNA from the transduced cells is captured with single cell technology in step (d).

120. The method of claim 118 or 119, wherein the method further comprises determining the level of mRNA expression and/or the level of protein expression in the transduced cells.

121. The method of claim 120, wherein the method further comprises:
grouping transduced cells having the same gRNA; and
comparing the target gene expression of transduced cells having the same gRNA, at the mRNA and/or protein level, to the target gene expression of cells without the same gRNA.

122. The method of any one of claims 118-121, further comprising identifying the target gene of the gRNA sequenced in step (e).

123. The method of claim 122, further comprising modulating the level of the gene target or modulating the activity of the protein produced from the gene target for enhancing properties of a cell therapy.

124. The method of any one of claims 118-123, wherein the cell is an immune cell.

125. The method of claim 124, wherein the immune cell is a T cell.

126. The method of any one of claims 118-125, wherein the first polypeptide domain comprises a Staphylococcus aureus Cas9 protein (SaCas9).

127. The method of claim 126, wherein the first polypeptide domain comprises a nuclease-inactivated Staphylococcus aureus Cas9 protein (dSaCas9).
